(19) United States
(12) Patent Application Publication
(10) Pub. No.: US 2026/0343183 A1
(43) Pub. Date: Oct. 29, 2026

(54) SYSTEMS AND METHODS FOR LOCATING MOBILE TAGS IN REAL-TIME IN A MEDICAL FACILITY

(71) Applicant: UMANO MEDICAL INC., L'Islet (CA)

(72) Inventors: Anouer KEBIR, Levis (CA); Esther BERTHELOT, Cap d'Espoir (CA); David MAYEN MORENO, L'Ancienne-Lorette (CA); Ali DUFOUR, St-Augustin-De-Desmaures (CA)

(73) Assignee: UMANO MEDICAL INC., L'Islet (CA)

(21) Appl. No.: 19/462,167

(22) Filed: Jan. 28, 2026

Related U.S. Application Data
(60) Provisional application No. 63/796,697, filed on Apr. 29, 2025.

(51) Int. Cl. H04W 4/029; G16H 40/40; G16H 40/67

(52) U.S. Cl. CPC H04W 4/029; G16H 40/40; G16H 40/67

(57) ABSTRACT

There are provided systems, methods, patient support apparatuses and non-transitory storage mediums for locating mobile tags in real-time in a medical facility where the tags could be worn by patients or attached to equipment. Wireless node may acquire wireless signal parameters such as signal strength from wireless signals transmitted by tags. A real-time location system (RTLS) engine acquires the wireless signal parameters and estimates a distance between tags and wireless nodes. The estimated distances are used to classify the proximity of tags to wireless nodes.

### TECHNICAL FIELD

The present disclosure relates to systems, methods, patient support apparatuses and non-transitory storage mediums for locating mobile tags in real-time in a medical facility where the tags could be worn by patients or attached to equipment.

### BACKGROUND OF THE ART

Real-time localization technologies have been increasingly adopted in healthcare environments to support monitoring, tracking, and management of patients, beds, and medical equipment. These systems are generally implemented to maintain situational awareness within a clinical setting and to allow clinical and support staff to determine the physical location of assets and individuals within a defined space such as a hospital ward, intensive care unit, or surgical suite. Traditional methods for patient or equipment tracking have relied on manual data entry, visual inspection, or the use of fixed-location data collection points, which may not offer continuous or real-time positional awareness.

While such systems are available in various forms, the real-time synchronization of localization data across patients, beds, and mobile medical equipment remains subject to integration constraints and signal interference challenges in complex indoor clinical environments. Further, existing implementations may not fully address dynamic changes in room configurations, reallocation of beds, or context-aware associations between patient identity, bed occupancy, and the use of specific medical devices. Accordingly, improved systems and methods for real-time localization in hospital environments are under continued development to provide updated positional correlation across heterogeneous entities within a care facility.

There is a desire to locate equipment and patients in a medical facility.

### SUMMARY

It is an object of the present disclosure to ameliorate at least one drawback of existing systems.

It is an object of one or more implementations of the present disclosure to provide systems, methods, patient support apparatuses and non-transitory storage mediums for locating mobile tags in real-time in a medical facility, where the tags could be worn by patients or attached to equipment.

One or more embodiments of the present disclosure enable at least approximately locating mobile tags via wireless locating nodes without the use of pre-established signal calibration data or pre-mapped tag location information. The mobile tags may be equipped with transmitters and/or receivers of one or more types of wireless signals, such as, but not limited to, infrared (IR), Bluetooth including Bluetooth Low Energy (BLE), ultrawideband (UWB) and/or radiofrequency identification (RFID). Depending on the type of wireless technology used, the mobile tags may be located by a location engine via the wireless locating nodes, either directly (e.g., by determining wireless signal parameters such as signal strength) or indirectly (e.g., via patient support apparatuses detecting the mobile tags using proximity sensing, and the patient support apparatuses being located by the wireless locating nodes using wireless signals).

One or more other embodiments of the present disclosure aim to train machine learning models to detect presence of mobile tags within a zone or radius by using calibration data of the wireless signals and at least an approximate mapping of the areas in which the mobile tags are located.

One or more embodiments of the present disclosure enable reuse of the existing real-time location system (RTLS) infrastructure, wherein the wireless locating nodes or terminals currently installed could be operable without requiring relocation or hardware modification and by performing software updates. One or more embodiments of the present disclosure further enable a reduction in development costs through the use of standard tags (e.g., iBeacon tags), thereby avoiding hardware development expenditures. By employing existing and widely available beacons, financial investments typically associated with the design and manufacturing of proprietary components are not incurred. One or more embodiments of the present disclosure also provides a localization configuration with increased flexibility by using mobile tags available in multiple formats, including compact adhesive discs configured for attachment to various objects, adaptable bracelets, cards, or blocks. These available formats for mobile tags according to the present disclosure enable the system to be adapted to different localization requirements in hospital environments while maintaining flexibility of implementation.

At least some embodiments of the present technology enable locating tags (which may be worn by patients and/or attached to objects) by estimating the distance using the wireless signal strength data determined by wireless nodes in the facility, and by classifying the estimated distance between nodes and tags into two or more proximity categories, such as very close, close and far.

In one or more alternative embodiments, tags may be located by estimating the distance using non-power based wireless signal parameters, such as signal propagation time measurements or phase difference measurement across multiple frequencies determined by the wireless nodes in the facility.

In accordance with a broad aspect of the present technology, there is provided a system for locating a mobile tag in real-time in a medical facility, the system comprising: at least one mobile tag, the at least one mobile tag being configured for: transmitting a first type of wireless signal comprising a unique identifier, a set of wireless locating nodes, each wireless locating node being positioned at a respective location in the medical facility, each wireless locating node being configured for: receiving the first type of wireless signal, determining, based on the received first type of wireless signal, respective first signal parameters of the at least one mobile tag, and at least one computing device, the at least one computing device being connected to the set of wireless locating nodes, the at least one computing device being configured for: receiving respective first signal parameters of the first type of wireless signal transmitted by the at least one wireless mobile tag and received by each respective wireless locating node from the at least one mobile tag, determining, based the respective first signal parameters, a respective signal strength of the at least one mobile tag for each respective wireless locating node, estimating, based on the respective signal strengths, respective distances between the at least one wireless mobile tag and each respective wireless locating node, and classifying, based on the respective estimated distances, a respective proximity of the at least one wireless mobile tag to each respective wireless locating node.

In one or more embodiments of the system, the respective first signal parameters comprise a respective Received Signal Strength Indicator (RSSI).

In one or more embodiments of the system, said determining the respective signal strength for each respective wireless locating node from the at least one mobile tag comprises: applying a Kalman filter on the respective RSSI to obtain the respective signal strength.

In one or more embodiments of the system, said estimating, based on the respective signal strength, the respective proximity comprises: calculating the respective proximity based on at least the respective signal strength and a path loss exponent of an environment.

In one or more embodiments of the system, each wireless locating node of the set of wireless locating nodes is further configured for: transmitting a second type of wireless signal comprising a respective indication of a respective wireless node identifier, the at least one wireless mobile tag is further configured for: receiving, from a given wireless node of the set of wireless nodes, a second type of wireless signal comprising an indication of the given wireless node identifier, transmitting in the first type of wireless signal, the indication of the given wireless node identifier, the at least one computing device is further configured for: determining, based on the respective proximities and the given wireless node identifier, a location of the given wireless node.

In one or more embodiments, the second type of wireless signal has limited propagation through physical barriers, which enables determining presence of the at least one mobile tag within a specific room.

In one or more embodiments of the system, the first type of wireless signal comprises a Bluetooth signal and the second type of wireless signal comprises an infrared signal.

In one or more embodiments of the system, the first type of wireless signal comprises a Bluetooth signal and the second type of wireless signal comprises an ultrasound signal.

In one or more embodiments of the system, the at least one mobile tag is integrated into a wristband.

In one or more embodiments of the system, the at least one mobile tag is attachable to an article of clothing.

In one or more embodiments of the system, the at least one mobile tag further comprises an accelerometer, the at least one mobile tag being configured for transmitting accelerometer motion data.

In one or more embodiments of the system, the at least one computing device is further configured for: receiving the accelerometer motion data, and detecting, based on the accelerometer motion data, a position of the patient.

In accordance with a broad aspect of the present technology, there is provided a system for locating a mobile tag in real-time in a medical facility, the system comprising: at least one mobile tag, the at least one mobile tag being configured for: transmitting a first type of wireless signal comprising a unique identifier, a set of wireless locating nodes, each wireless locating node being positioned at a respective location in the medical facility, each wireless locating node being configured for: receiving the first type of wireless signal, determining, based on the received first type of wireless signal, respective first signal parameters of the at least one mobile tag, and at least one computing device, the at least one computing device being connected to the set of wireless locating nodes, the at least one computing device being configured for: receiving respective first signal parameters of the first type of wireless signal transmitted by the at least one wireless mobile tag and received by each respective wireless locating node from the at least one mobile tag, estimating, based on the respective first signal parameters, respective distances between the at least one wireless mobile tag and each respective wireless locating node, and classifying, based on the respective estimated distances, a respective proximity of the at least one wireless mobile tag to each respective wireless locating node.

In one or more embodiments, the respective first signal parameters of the first type of wireless signal comprise one of: signal propagation time measurement parameters and phase difference measurement across frequencies parameters.

In accordance with a broad aspect of the present technology, there is provided a method for approximately locating a wireless tag in a room of a facility, the method being executed by at least one processing unit, the at least one processing unit being connected to: a set of wireless locating nodes, each wireless locating node being positioned at a respective location in the facility, each wireless locating node being configured for transmitting and receiving a wireless signal, the at least one wireless mobile tag, the at least one wireless mobile tag being configured for receiving a wireless signal, the method comprising: receiving respective first signal parameters of the wireless signal transmitted by the at least one wireless mobile tag and received by each respective wireless locating node from the at least one mobile tag, determining, based the respective first signal parameters, a respective signal strength for each respective wireless locating node from the at least one mobile tag, estimating, based on the respective signal strengths, respective distances between the at least one wireless mobile tag and each respective wireless locating node, and classifying, based on the respective estimated distances, a respective proximity of the at least one wireless mobile tag to each respective wireless locating node.

In one or more embodiments of the method, the method further comprises outputting, to a user interface, the respective estimated distances and the respective proximity of the at least one wireless mobile tag to each respective wireless locating node.

In one or more embodiments of the method, the respective signal parameters comprise a respective Received Signal Strength Indicator (RSSI).

In one or more embodiments of the method, said determining the respective signal strength for each respective wireless locating node from the at least one mobile tag comprises applying a Kalman filter on the respective RSSI to obtain the respective signal strength.

In one or more embodiments of the method, said estimating, based on the respective signal strength, the respective proximity comprises: calculating the respective proximity based on at least the respective signal strength and a path loss exponent of the environment.

In one or more embodiments of the method, the respective signal parameters further comprises a respective RSSI at 1 meter, and said calculating the respective proximity is further based on the respective RSSI at 1 meter.

In one or more embodiments of the method, the respective proximity comprises one of: very close, close and far.

In one or more embodiments of the method, the wireless signal comprises a Bluetooth signal.

In one or more embodiments of the method, the method further comprises: associating, by using a rule-based procedure, based on the respective estimated distances and the respective proximity of the at least one wireless mobile tag to each respective wireless locating node, a given wireless node of the set of wireless nodes with the given wireless node.

In accordance with a broad aspect of the present technology, there is provided a method for predicting presence of a wireless tag in a room of a facility using a set of machine learning (ML) models, the method being executed by at least one processing unit, the at least one processing unit being connected to: a set of wireless locating nodes, each wireless locating node being positioned at a respective location in the facility, each wireless locating node being configured for transmitting and receiving wireless signals from a set of mobile tags, the at least one processing unit having access to the set of ML models, each ML model being associated with a respective wireless locating node of the set of wireless locating nodes, each ML model having been trained for predicting the presence of a given wireless tag, the method comprising: receiving, from each of the set of wireless locating nodes, respective first signal parameters from at least a subset of the mobile tags, extracting, for each of the set of wireless locating nodes, based on the respective first signal parameters, a respective set of features, determining, for each of the set of wireless locating nodes, based on the respective set of features, an indication of a presence of each mobile tag of the set of mobile tags in the room, and outputting, to a user interface, the indication of the presence of each mobile tag of the set of mobile tags in the room.

In one or more embodiments of the method, the method further comprises, prior to said receiving, from each of the set of wireless locating nodes, the respective first signal parameters from at least a subset of the mobile tags: training each ML model of the set of ML models associated with a respective wireless locating node of the set of wireless locating nodes on a set of respective first signal parameters for a set of positions of a second set of wireless tags in the facility, each wireless locating node being associated with a respective room, each of the second set of wireless tags being associated with a respective label, the respective label being indicative of the presence of the wireless in the respective room.

In one or more embodiments of the method, each ML model of the set of ML models is a neural network.

In accordance with a broad aspect of the present technology, there is provided a mobile tag comprising: a first receiver configured for receiving a first type of wireless signal, a second transceiver configured for transmitting a second type of wireless signal, a processing unit operatively connected to the first transceiver and the second transceiver, the processing unit being configured for: receiving the first type of wireless signal, in response to receiving the first type of wireless signal: transmitting, via the second transceiver, the second type of wireless signal, the second type of wireless signal comprising a unique identifier of the mobile tag.

In one or more embodiments of the mobile tag, the first receiver comprises an infrared receiver.

In one or more embodiments of the mobile tag, the second transceiver comprises a Bluetooth transceiver configured to broadcast iBeacon signals.

In one or more embodiments of the mobile tag, the mobile tag further comprises: a third transceiver configured for receiving and transmitting a third type of signal, and the at least one processing unit is configured for: transmitting and receiving the third type of wireless signal with at least one other transceiver, and determining, using the third type of signal, a distance between the mobile tag and the at least one other transceiver, and the second type of wireless signal comprises the distance.

In one or more embodiments of the mobile tag, the third transceiver comprises an ultrawideband (UWB) transceiver.

In one or more embodiments of the mobile tag, the mobile tag further comprises at least one accelerometer, the at least one accelerometer being configured to detect acceleration forces.

Each of the methods described herein may be implemented as computer-readable instructions stored in a non-transitory storage medium, which may be executed by one or more processing units.

In the context of the present specification, a “server” is a computer program that is running on appropriate hardware and is capable of receiving requests (e.g., from electronic devices) over a network (e.g., a communication network), and carrying out those requests, or causing those requests to be carried out. The hardware may be one physical computer or one physical computer system, but neither is required to be the case with respect to the present technology. In the present context, the use of the expression a “server” is not intended to mean that every task (e.g., received instructions or requests) or any particular task will have been received, carried out, or caused to be carried out, by the same server (i.e., the same software and/or hardware); it is intended to mean that any number of software elements or hardware devices may be involved in receiving/sending, carrying out or causing to be carried out any task or request, or the consequences of any task or request; and all of this software and hardware may be one server or multiple servers, both of which are included within the expressions “at least one server” and “a server”.

In the context of the present specification, “electronic device” is any computing apparatus or computer hardware that is capable of running software appropriate to the relevant task at hand. Thus, some (non-limiting) examples of electronic devices include general purpose personal computers (desktops, laptops, netbooks, etc.), mobile computing devices, smartphones, and tablets, and network equipment such as routers, switches, and gateways. It should be noted that an electronic device in the present context is not precluded from acting as a server to other electronic devices. The use of the expression “an electronic device” does not preclude multiple electronic devices being used in receiving/sending, carrying out or causing to be carried out any task or request, or the consequences of any task or request, or steps of any method described herein. In the context of the present specification, a “client device” refers to any of a range of end-user client electronic devices, associated with a user, such as personal computers, tablets, smartphones, and the like.

In the context of the present specification, the expression “computer readable storage medium” (also referred to as “storage medium” and “storage”) is intended to include non-transitory media of any nature and kind whatsoever, including without limitation RAM, ROM, disks (CD-ROMs, DVDs, floppy disks, hard drivers, etc.), USB keys, solid state-drives, tape drives, etc. A plurality of components may be combined to form the computer information storage media, including two or more media components of a same type and/or two or more media components of different types.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. Thus, information includes, but is not limited to audiovisual works (images, movies, sound records, presentations etc.), data (location data, numerical data, etc.), text (opinions, comments, questions, messages, etc.), documents, spreadsheets, lists of words, etc.

In the context of the present specification, unless expressly provided otherwise, an “indication” of an information element may be the information element itself or a pointer, reference, link, or other indirect mechanism enabling the recipient of the indication to locate a network, memory, database, or other computer-readable medium location from which the information element may be retrieved. For example, an indication of a document could include the document itself (i.e. its contents), or it could be a unique document descriptor identifying a file with respect to a particular file system, or some other means of directing the recipient of the indication to a network location, memory address, database table, or other location where the file may be accessed. As one skilled in the art would recognize, the degree of precision required in such an indication depends on the extent of any prior understanding about the interpretation to be given to information being exchanged as between the sender and the recipient of the indication. For example, if it is understood prior to a communication between a sender and a recipient that an indication of an information element will take the form of a database key for an entry in a particular table of a predetermined database containing the information element, then the sending of the database key is all that is required to effectively convey the information element to the recipient, even though the information element itself was not transmitted as between the sender and the recipient of the indication. As a non-limiting example, to “display an indication” involves rendering, through execution of instructions, at least one visual element representing the information element, which may be the information element itself (e.g., value or text) or a graphical element representing the information element (e.g. progress bar or color for intensity values).

In the context of the present specification, the expression “communication network” is intended to include a telecommunications network such as a computer network, the Internet, a telephone network, a Telex network, a TCP/IP data network (e.g., a WAN network, a LAN network, etc.), and the like. The term “communication network” includes a wired network or direct-wired connection, and wireless media such as acoustic, radio frequency (RF), infrared and other wireless media, as well as combinations of any of the above.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “first server” and “third server” is not intended to imply any particular order, type, chronology, hierarchy or ranking (for example) of/between the server, nor is their use (by itself) intended to imply that any “second server” must necessarily exist in any given situation. Further, as discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes which may be substantially represented in computer-readable media and so executed by a computer or processor, whether or not such computer or processor is explicitly shown.

The functions of the various elements described herein and/or shown in the figures, including a “processor”, “processing unit”, “graphics processing unit” or the like may be provided through the use of dedicated hardware as well as hardware capable of executing software in association with appropriate software. When provided by a processing unit, the functions may be provided by a single dedicated processor, by a single shared processor, or by a plurality of individual processors, some of which may be shared. In some non-limiting embodiments of the present technology, the processing unit may be a general purpose processor, such as a central processing unit (CPU) or a processor dedicated to a specific purpose, such as a graphics processing unit (GPU). Moreover, explicit use of the term “processor” or “controller” should not be construed to refer exclusively to hardware capable of executing software, and may implicitly include, without limitation, digital signal processor (DSP) hardware, a microcontroller, network processor, an integrated circuit (IC) purposed for specific operations within an embedded system, application specific integrated circuit (ASIC), field programmable gate array (FPGA), a system on a chip (SoC), and the like.

The functions of the various elements described herein and/or shown in the figures including “memories” may include volatile and non-volatile computer-readable storage media realized in semiconductor, magnetic, optical, or other physical forms and may reside locally, be distributed across multiple devices, or be accessed through a network-based storage service. In particular implementations, a given one of the memories comprises volatile storage such as random-access memory (RAM), static RAM (SRAM), or dynamic RAM (DRAM), and/or non-volatile storage such as flash memory, electrically erasable programmable read-only memory (EEPROM), ferroelectric RAM (FRAM), magnetic disk, optical disk, or solid-state drive. The memories may be removable or non-removable, may interface with the processors through one or more buses or interconnects employing standard or proprietary protocols, and are configured to store computer-readable instructions, data structures, configuration parameters, machine-learning models, and other information processed by the processors to carry out the functionalities described herein.

Software modules, or simply modules which are implied to be software, may be represented herein as any combination of flowchart elements or other elements indicating performance of process steps and/or textual description. Such modules may be executed by hardware that is expressly or implicitly shown.

### BRIEF DESCRIPTION OF THE DRAWINGS

Having thus generally described the nature of the technology, reference will now be made to the accompanying drawings, showing by way of illustration example embodiments thereof and in which:

FIG. 1 is a perspective view of a patient support apparatus in accordance with one or more non-limiting embodiments of the present disclosure;

FIG. 2 illustrates a schematic diagram of a real-time localization communication system 200 in accordance with one or more non-limiting embodiments of the present disclosure;

FIG. 3 illustrates a schematic diagram of an environment implementing an RTLS system in a medical facility in accordance with one or more non-limiting embodiments of the present disclosure;

FIG. 4 illustrates a schematic diagram of an environment in a medical facility where different locations of mobile tags attached to patients or equipment are shown in accordance with one or more non-limiting embodiments of the present disclosure;

FIG. 5 illustrates a schematic diagram of an environment for measuring signals for generating training data in accordance with one or more non-limiting embodiments of the present disclosure;

FIG. 6 is a flow chart of steps of a first procedure for locating mobile tags in accordance with one or more non-limiting embodiments of the present disclosure;

FIG. 7 is a flow chart of main steps of a procedure for connecting to a message queuing telemetry transport (MQQT) broker in accordance with one or more non-limiting embodiments of the present disclosure;

FIG. 8 is a flow chart of steps of a procedure for receiving iBeacon messages in accordance with one or more non-limiting embodiments of the present disclosure;

FIG. 9 is a flow chart of steps of a procedure for filtering the received signal strength indicator (RSSI) with a Kalman Filter in accordance with one or more non-limiting embodiments of the present disclosure;

FIG. 10 is a flow chart of steps of a procedure for calculating an estimated distance based on the RSSI in accordance with one or more non-limiting embodiments of the present disclosure;

FIG. 11 is a flow chart of steps of a procedure for proximity classification based on the estimated distance in accordance with one or more non-limiting embodiments of the present disclosure;

FIG. 12 includes FIG. 12A, FIG. 12B and re FIG. 12C which when combined form flow charts of steps of a procedure for applying decision rules in accordance with one or more non-limiting embodiments of the present disclosure;

FIG. 13 is a flow chart of steps of a period processing procedure for mobile tags in accordance with one or more non-limiting embodiments of the present disclosure;

FIG. 14 includes FIG. 14A and FIG. 14B which when combined form a flow chart of steps of a procedure for locating mobile tags using infrared and Bluetooth signals in accordance with one or more non-limiting embodiments of the present disclosure;

FIG. 15 illustrates a flowchart of a method of locating wireless mobile tags using power-based signal parameters in accordance with one or more non-limiting embodiments of the present disclosure;

FIG. 16 illustrates a flowchart of a method of locating wireless mobile tags using one of signal propagation time measurement parameters or phase difference measurement across frequencies parameters in accordance with one or more non-limiting embodiments of the present disclosure;

FIG. 17 illustrates a flowchart of a method of locating wireless mobile tags using a set of trained machine learning models in accordance with one or more non-limiting embodiments of the present disclosure; and

FIG. 18 is an image of a wireless tag integrated into a wristband in accordance with one or more non-limiting embodiments of the present disclosure.

### DETAILED DESCRIPTION

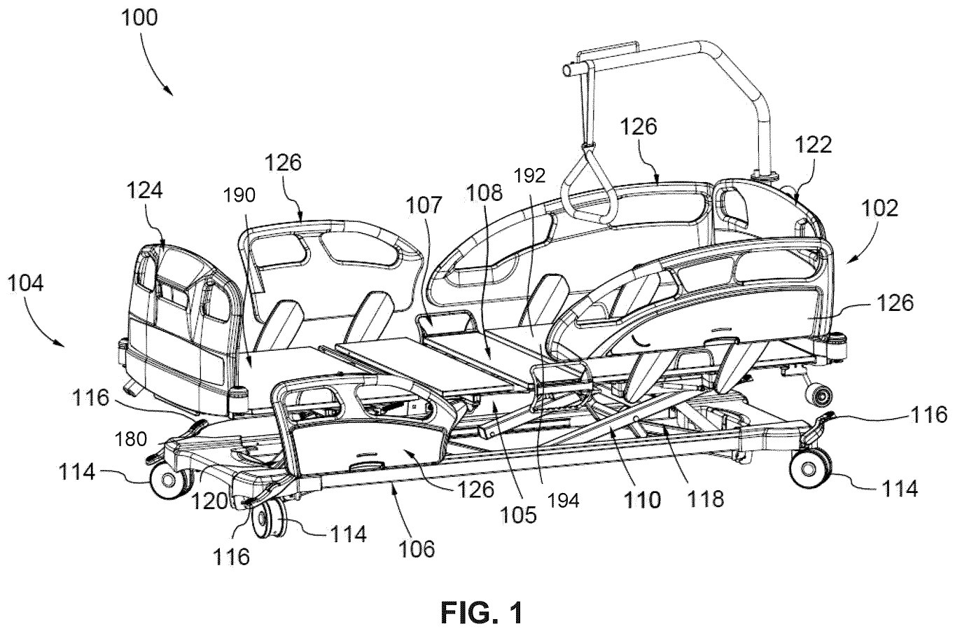

A patient support apparatus 100 in accordance with an embodiment of the present technology is illustrated in FIG. 1. The patient support apparatus 100 may be used in a medical setting for supporting a patient. In this embodiment, the patient support apparatus 100 is a hospital bed 100 that is used in a hospital, and in particular in an intensive care unit (ICU) or medical-surgical (med-surg) setting. It is contemplated that, in other embodiments, the patient support apparatus 100 may be a different type of patient support apparatus such as, for example, a stretcher, a motorized chair, an operating room table, or other specialty tables (e.g., an examination table) used in different contexts.

With reference to FIG. 1, the bed 100 has a head end 102 and a foot end 104 opposite each other and defining a length of the bed 100 therebetween. As will be appreciated, in use, when the patient is lying on the bed 100, the patient's head is closer to the head end 102 while the patient's feet are closer to the foot end 104. The bed 100 also has left and right sides 105, 107 extending between the head end 102 and the foot end 104 and defining a width of the bed 100 therebetween.

Some of the structural components of the bed 100 will be designated hereinafter as “right”, “left”, “head” and “foot” from the reference point of an individual lying on their back on the bed 100 with their head oriented toward the head end 102 of the bed 100 and their feet oriented toward the foot end 104 of the bed 100. Similarly, the term “headward” refers to an element located towards the head end 102 of the bed 100 and the term “footward” refers to an element located towards the foot end 104 of the bed 100. Furthermore “interior” and “exterior” views are also designated from the reference point of the patient lying in the bed. Therefore, an interior view shows an element as seen by the patient looking toward the environment outside of the bed and an exterior view shows an element as seen by a person outside of the bed. Generally, an exterior view shows the exterior surfaces of the bed, and the interior view shows the interior surfaces of the bed. The terms “inner” and “outer” will similarly be used to describe the position of elements relative to the bed.

The bed 100 has a base 106 and a patient support assembly 108 operatively connected to the base 106. Four casters 114 are connected to the base 106 at respective corners thereof to allow the bed 100 to be moved along a floor. Additional casters may be provided in other embodiments. Respective brakes 116 are provided for each caster 114 to selectively lock and unlock the casters 114. The bed 100 may also have a drive wheel (not shown) connected to the base 106 for driving the bed 100 on the floor.

The patient support assembly 108, which is disposed above the base 106, is configured to accommodate the patient thereon and includes an upper frame 180 and a deck 190 supported by the upper frame 180. The upper frame 180 extends from a head end 201 to an opposite foot end 203. The upper frame 180 has left and right longitudinal rails 202 that extend parallel to each other and are interconnected by a plurality of transversal members 204a, 204b, 204c extending therebetween. The upper frame 180 may be configured in any suitable manner in other embodiments.

The deck 190 defines a top face on which a support surface such as a mattress or the like is supported. Deck 190 includes a plurality of deck sections that are connected to the frame 180 and, in use, support different areas of the patient's body. Notably, the deck sections forming the deck 190 include an upper body deck section (not numbered) for supporting an upper body of the patient, a seat deck section (not numbered) for supporting the patient's upper legs area, a thigh deck section (not numbered) for supporting the patient's thighs, and a foot deck section (not numbered) for supporting the patient's lower legs and feet. The deck sections (not numbered) are longitudinally consecutive to each other and together form the top face. Notably, the upper body deck section, seat deck section, thigh deck section and foot deck section have an upper body deck section panel, a seat deck section panel, a thigh deck section panel and a foot deck section panel, respectively. The deck section panels define respective top faces such that, together, they form the top face 192 of the deck 190.

The deck sections can be articulated in such a manner as to optimize comfort for the patient lying on the bed 100. The deck sections are movably connected to each other. Example embodiments for the articulation of deck sections are known in the art and some are described in greater detail in International Patent Application Publication No. WO2024154074, by the same Applicant, the entirety of which is incorporated by reference herein. It is contemplated that fewer deck sections may be provided in other embodiments (e.g., three deck sections).

The bed 100 has a plurality of barriers generally disposed around the patient support assembly 108 including endboards, namely a headboard 122 at the head end 102 and a footboard 124 at the foot end 104, and at least one siderail 126 on the left and right sides 105, 107. The siderails 126 can be raised to prevent the patient from falling off the bed 100 or lowered (as shown for one of the siderails 126 in FIG. 1) to allow the patient to exit the bed 100 and/or to allow care providers to access and attend to the patient. In the embodiment shown in FIG. 1, the siderails 126 include two siderails on each side (a head siderail and a foot siderail).

An elevation system 110 operatively connects the patient support assembly 108 to the base 106 to move the patient support assembly 108 relative to the base 106, namely selectively raising or lowering the patient support assembly 108 relative to the base 106. In addition to raising and lowering the patient support assembly 108, the elevation system 110 also allows the bed 100 to assume various different positions such as a flat horizontal position (shown in FIG. 1), a Trendelenburg position, a reverse Trendelenburg position, a cardiac chair position and a full chair position. It is contemplated that, in other embodiments, the bed 100 may be configured to assume more or fewer positions. For instance, in some cases, the bed 100 may permanently remain in the flat horizontal position and simply be raised and lowered by the elevation system 110.

The base 106 further comprises force sensors for determining forces exerted on the bed 100. In one example embodiment, a plurality of load cells are adapted to provide an indication of the weight on the bed 100. The one or more load cells are operatively connected to a controller. The force sensors may also be used for determining a position of the patient in the bed 100, a center of mass of the weight present on the bed, a level of activity of the patient and a load distribution of the weight of the patient.

In one or more embodiments, the bed 100 includes four load cells, where each load cell is positioned at a respective corner i.e., foot right (FR), foot left (FL), head right (HR) and head left (HL). Each load cell is operable to generate a signal comprising a voltage value upon presence of a weight on the bed, which is converted into a respective weight value for each load cell received by the controller. The respective weight values may be used to determine the weight of the person or the object(s) on the bed 100, a position and/or movement of the center of gravity on the bed 100, and/or further processed to determine patient agitation and activity, vital signs (e.g., heart rate (HR) and respiratory rate (BR)) and the like. In one or more alternative embodiments, the bed 100 may include a different number of load cells, such as a single load cell.

The bed 100 includes one or more controllers (shown schematically as controller 180 in FIG. 1). In some embodiments, the bed 100 may include a bed controller and a mattress controller (not shown).

The controller 180 is operatively connected to various systems, subsystems and components of the hospital bed 100, which may include, as a non-limiting example, adjustment mechanisms, actuators, sensors, integrated weighing systems, alert indicators, and the like. In one or more embodiments, the controller 180 may be integrated or attached to the hospital bed 100.

The controller 180 comprises one or more processing unit(s), one or more memories, input/output interfaces and communication interfaces including one or more transceivers (not shown). It will be appreciated that the controller 180 is an embodiment of a computing device.

The one or more communication interfaces may include wired and wireless communication interfaces to connect the components of the hospital bed 100 to other medical devices, computing devices (e.g., wireless modules, nurse station computer, server(s) and mobile devices), and communication networks (e.g., hospital network or cellular network) to transmit an/or receive data.

The one or more communication interfaces may include wired and wireless communication interfaces to connect components of the hospital bed 100 to other medical devices, computing devices (e.g., nurse station computer, server(s) and mobile devices), and communication networks (e.g., hospital network) to transmit and receive data.

The one or more communication interfaces (not shown) may include wireless communication modules configured to transmit and receive one or more types of wireless signals (e.g., Wi-Fi, Bluetooth™ (including Bluetooth Low Energy (BLE)), Zigbee, cellular/GSM, Infrared (IR), radiofrequency (RF), ultrawide band (UWB), Near Field Communication (NFC), Radio-Frequency Identification (RFID), etc.) and wired communication modules (e.g., ethernet, USB, Serial, fiber optics, etc.). It will be appreciated that in some embodiments, at least a portion of the communication interfaces may be provided within a housing that may be removably secured to the bed.

The hospital bed 100 may optionally perform monitoring functions. For example, weight sensors (not shown) in a plurality of locations under the mattress (not shown) can be used to monitor for patient movement, as well as to determine the patient's location within the hospital bed 100 or whether the patient has left the hospital bed 100. In one example, the weight sensors may be integrated into the wheels of the hospital bed 100. In another example, one or more pressure sensors, such as pressure-sensitive mats sold under the Velostat™ name, may be disposed on one or more body panels 120 under the patient area of the hospital bed 100. In another example, the location of the patient can be determined by the use of a camera (not shown), for example an infrared (IR) or visible light camera or other type of sensor, mounted in any convenient location in the room and directed at the patient area of the bed.

The one or more communication modules (not shown) of the hospital bed 100 may be configured for communicating with other sensors that may or may not be attached to the patient, and may be configured for communicating sensor information to computing devices in the hospital so that it can be accessed by medical personnel. The hospital bed 100 may have additional features and capabilities, which may vary depending on the particular model of bed.

In one or more embodiments, the different systems, subsystems, components and sensors of the hospital bed 100 are connected via one or more databuses (e.g. via a Local Interconnect Network (LIN) bus) to transmit information to and/or receive information from the controller 180.

The controller 180 is operatively connected to one or more control panels (not shown). The control panel, also referred to as control interface, user interface, and input device, enables a user (e.g. caregiver) to interact with the different components of the hospital bed 100 and/or support surfaces (e.g. inflatable mattress) by displaying information and transmitting instructions to control different components and functions of the hospital bed and/or mattress.

The control panel may be mounted on, integrated into or may be adjacent to the hospital bed. It will be appreciated that one or more control panels with different components may be mounted on siderails, endboard (headboard, footboard) and/or fixed or removable panels of the hospital bed 100. It will be appreciated that control panels may be provided at different locations, may be integrated into the or may be a separate device operatively connected to at least one component of the bed.

The bed 100 has many other features that will not be described in detail herein as they are well known in the art. The above description of the bed is thus not meant to be exhaustive but rather to provide sufficient context for the reader.

Non-limiting examples of implementations of the hospital bed 100 include ook snow, ook snow ALL, ook snow MH, and ook cocoon manufactured by Umano Medical Inc. of Quebec, Canada.

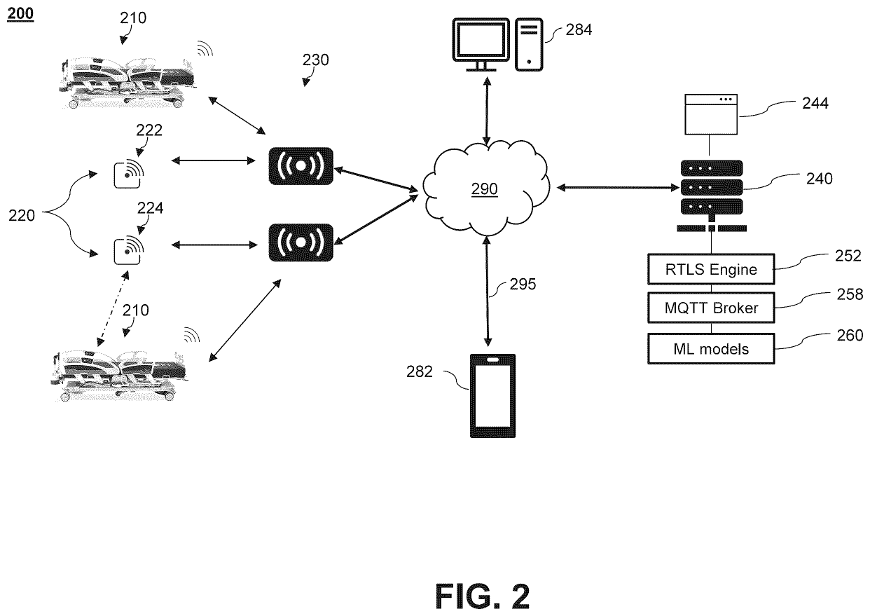

FIG. 2 illustrates a schematic diagram of a real-time localization communication system 200 suitable for implementing one or more embodiments of the present technology.

The real-time localization communication system 200 comprises a plurality of patient support apparatuses 210, a plurality of mobile wireless tags 220, a plurality of wireless locating nodes 230, a workstation computer 284, one or more client devices 282, and one or more servers 240 connected over a communication network 290 via respective communication links 295.

### Patient Support Apparatuses

The plurality of patient support apparatuses 210 (not separately numbered) may be located in a healthcare/medical facility. A given patient support apparatus 210 may be for example located within a zone of a room, a hallway, an intensive care unit, an emergency room, an operating room, and the like. The patient support apparatus 210 could be used in various locations without departing from the scope of the present technology.

A given patient support apparatus 210 includes communication interfaces with one or more transmitters and/or receivers configured to transmit and/or receive various types of wireless signals. In one or more embodiments, the different types of wireless signals may include Wi-Fi, Bluetooth, ultrawideband (UWB), infrared (IR), ultrasound, and RFID signals.

As a non-limiting example, a patient support apparatus may transmit and/or receive UWB and IR signals which may be used by the plurality of wireless locating nodes 230 for precisely locating the patient support apparatus.

In some embodiments, the one or more transmitters and/or receivers may be provided within a computing device or tag attached to a given patient support apparatus 210. As a non-limiting example, the tag may be removable and may be operatively connectable to a controller of the patient support apparatus 210.

In one or more other embodiments, the one or more transmitters and/or receivers may be integrated to the circuitries of the patient support apparatus.

In one or more alternative embodiments, a given patient support apparatus may include one or more RFID readers which enable to detect RFID within a detection range of the RFID reader. As a non-limiting example, a given patient support apparatus may include RFID readers that may detect wristbands with RFID transponders when the patient occupies the bed. It will be appreciated that other configurations may be possible.

### Mobile Tag

The real-time localization communication system 200 comprises the plurality of mobile wireless tags 220, which may also be referred to as portable identification devices (PID), mobile tags, beacons or transponders. In FIG. 2, the plurality of mobile wireless tags 220 includes two mobile wireless tags 222, 224, however it should be noted that the plurality of mobile wireless tags 220 can include dozens, hundreds, or more wireless tags without departing from the scope of the present technology.

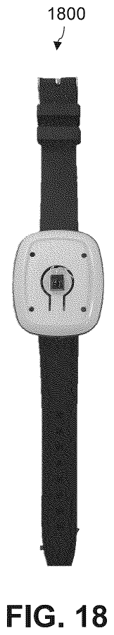

Each of the plurality of mobile wireless tags 220 is a device configured for portable use within an environment and operable to transmit and/or receive one or more types of wireless signals, which enables at least approximately locating the wireless tag. In one or more embodiments, a given mobile wireless tag may be attached to equipment (e.g. pumps, patient monitoring devices, ventilator, bedside diagnostic equipment, carts, etc.), objects (e.g., containers, fluid bags) and/or may be carried by a patient (e.g., in the form of a wearable badge, card, attached to a clothing piece, or a wearable device such as a wristband). A non-limiting example of a wireless tag in a wristband 1800 is shown in FIG. 18.

Each wireless tag 222, 224 of the plurality of wireless tags 220 is associated with a respective unique identifier (unique ID). A given wireless tag may include one or more radiofrequency transmitters and/or receivers. It should be understood that the number and types of wireless technologies used is not limited and may be combined. For example, the first wireless tag 222 may use Bluetooth and optionally IR transceivers, while the second wireless tag 224 may use RFID transceivers, and a third wireless tag (not shown) may use IR, UWB and Bluetooth transceivers without departing from the scope of the present technology. The term “transceiver” as used herein should be understood to encompass either a transmitter or a receiver, or as a combination transmitter-receiver.

In one or more embodiments, a given wireless tag 222, 224 is implemented as an iBeacon tag that periodically transmits its unique identifier via Bluetooth. As a non-limiting example, the iBeacon mobile tag may use Bluetooth low energy (BLE) to broadcast unique identifiers (e.g., MAC ID). It will be appreciated that the given wireless tag 222, 224 may transmit further information such as signal strength related parameters (e.g., RSSI at 1 m) if applicable.

In embodiments where a given wireless tag 222, 224 includes an UWB transceiver, the given wireless tag 222, 224 may be configured to determine a distance between the given wireless tag and a wireless locating node, patient support apparatus or other type of device configured with an UWB transceiver and processing capabilities. The distance may be determined using two-way ranging techniques or time difference of arrival (TDoA). The determined distance may then be transmitted via another type of wireless signal (e.g. Bluetooth).

It will be appreciated that alternatively, the distance may be determined by the other device with the UWB transceiver and processing capabilities (e.g. wireless locating node) instead of the given wireless tag with the UWB transceiver.

In one or more other embodiments, a given wireless tag 222, 224 may include an infrared receiver for confirming presence of the given wireless tag within an area (e.g. room), and a Bluetooth transceiver configured for transmitting and receiving BLE iBeacon signals. In one or more alternative embodiments, a given wireless tag 222, 224 may include ultrasonic transceivers for confirming presence of the given wireless tag within an area (e.g. room) using ultrasonic waves, and a Bluetooth transceiver configured for transmitting and receiving BLE iBeacon signals.

It will be appreciated that the second type of wireless signal (e.g., infrared or ultrasonic signal) has limited propagation through physical barriers, which enables determining presence of the at least one mobile tag within a specific room.

In one or more embodiments, the given wireless tag 222, 224 may be implemented using Bluetooth (e.g., iBeacon), UWB, and infrared technologies. In such embodiments, infrared may be used to detect presence of the wireless within an area (e.g., room), UWB may be used to determine the distance with cm precision, and BLE may be used to transmit the distance to the wireless locating node.

In some embodiments, a given wireless tag 222, 224 may also be associated with information such as product or asset information, sensor data, timestamp information, location information (if at a fixed location), status or condition data, authentication or security data, configuration parameters, and usage history or logs. As a non-limiting example, a given wireless tag may comprise an accelerometer to detect different types of movements and/or falls.

In one or more other embodiments, a given wireless tag 222, 224 may be a passive tag comprising one or more radio-frequency identification (RFID) transponders. As a non-limiting example, the given wireless tag may be attached to clothes worn by the patient or may be worn by the patient (e.g., on a wristband).

In some embodiments, the given wireless tag 222, 224 may include an accelerometer configured to detect acceleration forces along one or more axes (e.g., x; y; z). The accelerometer generates output signals representative of changes in velocity or orientation of the given wireless tag relative to a reference frame. The output signals may be processed to determine movement characteristics or positional information of the patient or the medical equipment to which it is attached. In such embodiments, the accelerometer motion data may be used to determine if the given wireless tag is in movement when the distance and/or the proximity of the given wireless tag to the terminal is determined, which may be used to adjust the determined distance or proximity (e.g., if the patient/equipment with the mobile tag is moving quickly) and/or provide an indication that the mobile tag is in movement to a user viewing the location.

As a non-limiting example, the given wireless tag 222, 224 including the accelerometer may be attached to the torso of the patient, and the acceleration data may be used to determine a position of the patient (e.g., lying down on their back, sitting, standing, ambulating, reclining, bending, side-lying, seated upright, slouched) as well as transitions between positions including falls. In some implementations, the accelerometer may be implemented as a Microelectromechanical Systems (MEMS) and may further configured to detect vital signs of the patient.

The given wireless tag may communicate the data to a computing device (e.g., server 240) using the transceivers (e.g. via Bluetooth). It will be appreciated that presence in the bed may be determined using the distance of the wireless tag from the bed/wireless locating node (e.g. via UWB), may be determined using confirmation from the bed load cells, or directly by the bed (in embodiments where the given wireless tag includes RFID technology and the bed an RFID reader).

In one or more embodiments of the present disclosure, a given wireless tag may be at least approximately located within a room, or its proximity to one or more patient support apparatuses, wireless terminals or other mobile tags may be determined, as will be explained hereinafter.

The real-time localization communication system 200 comprises the plurality of wireless locating nodes 230 (not separately numbered).

### Wireless Locating Node

The wireless locating node 230, which may also be referred to as a wireless terminal, wireless node, wireless anchor, headwall, wireless stationary node, or locator, is a computing device with components configured for transmitting and/or receiving a plurality of types of wireless signals for locating different types of mobile wireless tags attached to beds, equipment, personnel, visitors and/or patients.

The wireless locating node 230 comprises a housing with one or more processing units, one or more memories, and one or more communication interfaces including wireless antennas for transmitting and/or receiving the different types of wireless signals. A given wireless locating node may be positioned within a zone of a room for detecting patient support apparatuses and mobile tags. As a non-limiting example, a wireless locating node 230 may be fixed to a ceiling or a wall of a room, and a room with a plurality of beds may include a plurality of wireless locating nodes. A given wireless terminal may be associated with a unique wireless terminal ID.

In one or more embodiments, the wireless locating node 230 may include an ESP32 SoC microcontroller manufactured by Taiwan Semiconductor Manufacturing Company (TSMC) Limited from Hsinchu Science Park, Taiwan.

A given wireless locating node 230 may be operatively connected via a wired or wireless connection for exchanging data via different types of networks, such as local networks, nurse call networks, hospital networks, mobile networks and the Internet.

In one or more embodiments, the wireless locating node 230 may be used to at least approximately locate a given mobile wireless tag, and transmit the location data via the communication network 290.

In one or more embodiments, the wireless locating node 230 is configured for transmitting and/or receiving one or more of: Wi-Fi signals, infrared (IR) signals, ultrasonic signals, ultrawideband (UWB) signals and Bluetooth signals. Additionally, or alternatively, the wireless terminal may use other types of RF protocols including Zigbee, near-filed communication (NFC), cellular, and the like.

In one or more embodiments, Bluetooth, IR, UWB and/or ultrasonic signals may be used to locate personal identification devices (PID) such as tags or patient support apparatuses (e.g., hospital beds) equipped with IR and UWB tags (not shown in FIG. 2), as described in International Patent Publication WO 2022/208437 A1 by the same Applicant, which is herein incorporated by reference in its entirety.

As a non-limiting example, UWB signals may be used to determine distance with one of time difference of arrival (TDoA) and two-way ranging (TWR).

In one or more embodiments, Bluetooth signals may be used to locate mobile tags attached to medical equipment, personnel and/or patients. In one or more embodiments, a given wireless locating node may be configured for detecting wireless signals emitted by a given mobile tag and for determining wireless signal parameters including a received signal strength indicator (RSSI) and transmitting the wireless signal parameters to the server 240 executing the RTLS engine 252.

In one or more alternative embodiments, Bluetooth signals could be used to determine a distance between a given one of the plurality of wireless locating nodes 230 and a given mobile tag 222 using Bluetooth channel sounding methods. As a non-limiting example, the method may include transmitting, by the terminal, a sounding packet containing a known reference sequence over a designated Bluetooth channel. The given mobile tag 222 may receive the sounding packet and process the reference sequence to estimate a channel impulse response or phase information indicative of signal propagation characteristics. The mobile tag 222 generates a response packet including the channel estimation data and a timestamp corresponding to the time of reception, and transmits the response packet to the terminal. Upon receiving the response packet, the wireless node 230 calculates a time-of-flight or phase shift value based on the round-trip time and the reference signal characteristics, and determines an estimated distance between the terminal and the mobile tag using the speed of electromagnetic wave propagation and the computed timing or phase shift information. It will be appreciated that the distance may in some embodiments be determined by the given mobile tag 222, while in other embodiments it may be determined by another computing device.

In one or more other embodiments, a combination of Bluetooth signals and IR signals may be used to locate mobile tags attached to medical equipment, personnel, visitors and/or patients. In such embodiments, a given wireless locating node may be configured for: (i) transmitting infrared signals; (ii) detecting Bluetooth signals emitted by mobile tags including a given mobile tag having received the IR signal; (iii) determining wireless signal parameters including the received signal strength indicator (RSSI); and (iv) transmitting the wireless signal parameters to the server 240 executing the RTLS engine 252. It will be appreciated that this enables detecting tags in line of sight of the wireless locating node using IR.

### Server

The real-time localization communication system 200 comprises a server 240. While illustrated as a single server for simplicity, it should be understood that the functionality of the server 240 described herein may be distributed among a plurality of servers.

The server 240 is configured to inter alia: (i) execute a monitoring functionality 244; (ii) execute a real-time locating system (RTLS) engine 252; (iii) execute a Message Queuing Telemetry Transport (MQTT) broker functionality 258; (iv) execute a training procedure of ML models 260; (v) use the ML models 260 for inference.

It should be noted that the training procedure of ML models 260 and inference of ML models 260 does not need to be performed in each and every embodiment of the present technology.

In one or more embodiments, the RTLS engine 252 is configured to inter alia: (i) receive respective first signal parameters of a first type of wireless signal transmitted by at least one wireless tag and received by each respective wireless locating node from the at least one mobile tag; (ii) determine, based the respective first signal parameters, a respective signal strength for each respective wireless locating node from the at least one mobile tag; (iii) estimate, based on the respective signal strengths, respective distances between the at least one wireless tag and each respective wireless locating node; (iv) classify, based on the respective estimated distances, a respective proximity of the at least one wireless tag to each respective wireless locating node.

In one or more embodiments, the RTLS engine 252 is configured to use rule-based procedures to associate wireless locating nodes to respective tags based on the estimated distances and the classified proximities. Examples of rule-based procedures will be provided hereinafter.

The MQTT broker functionality 258 is configured to ensure the efficient transmission of data from the wireless nodes to the RTLS engine 252, by using the MQTT protocol. An MQTT broker is implemented to manage communication between the different clients of the system. The use of the MQTT protocol enables using a multiplicity of providers which reduces technological dependency, enables high transparency which facilitates adaptation to future requirements, and provides lightweight and optimized protocol specifically designed for Internet of Things (IoT) applications. The MQTT broker functionality 258 may be implemented as a microservice. In such embodiments, the server 240 (or another server) may act as an MQTT broker, while devices such as the wireless nodes may act as a client.

In the context of the present disclosure, the MQTT broker functionality 258 is used for transmitting and managing location information from the plurality of tags 220 and/or the plurality of wireless locating nodes 230.

In one or alternative embodiments, the MQTT broker functionality 258 may be replaced by one or more of. Apache Kafka, RabbitMQ, WebSocket, AWS IoT Core, Google Cloud IoT Core, ActiveMQ and REST APIs without departing from the scope of the present technology.

In one or more embodiments, the MQTT broker functionality 258 and the RTLS engine 252 may be provided by a single server. In one or more other embodiments, the MQTT broker functionality 258 and the RTLS engine 252 may be distributed among a plurality of servers.

In one or more embodiments, the server 240 is configured to provide a graphical user interface (GUI) via the monitoring functionality 244, which enables to receive and display data related to the subsystems and components of the patient support apparatus 210 (e.g., bed status, bed exit, alerts, etc.) and transmit control data to at least some of the subsystems and components of the patient support apparatus 210 via the GUI. In the context of the present disclosure, the monitoring functionality 244 provides monitoring data and location data of the plurality of patient support apparatuses 210, the plurality of mobile tags 220, and the plurality of wireless locating nodes 230. It will be appreciated that the GUI may be accessed by client devices such as client device 282 and workstation computer 284.

The server 240 is configured to process numerical data and transform the numerical data into multiple forms of visual representations. The visual representations may include, but are not limited to, line plots, bar graphs, pie charts, scatter plots, heat maps, histograms, and color-coded diagrams. Additionally, the server 240 is configured to generate visualizations like 2D and 3D graphs, time lapses, interactive plots, and real-time data visualizations. The generated visual data is then displayed as a part of the GUI, which can be accessed by users through computing devices and/or display units such as the nurse station computer 356, and the client device 282, for example via a monitoring application (not shown). The monitoring application 244 enables users to interact with the visual data, as a non-limiting example via functionalities like zooming, panning, and selecting specific data points for a more detailed view, or program functionalities of the patient support apparatus 210.

The workstation computer 284 is a centralized computing system configured to manage and access patient information, location data of the patient support apparatuses 210, the plurality of wireless nodes 230 and the plurality of mobile tags 220, coordinate care, and to enable and facilitate communication among healthcare professionals in a healthcare facility. The workstation computer 284 is configured to access and/or store electronic medical records (EMRs), scheduling and tracking patient appointments, integrating with hospital-wide communication and monitoring systems, assisting in medication management, and providing tools for reporting and analytics.

In one or more implementations of the present technology, the workstation computer 284 is configured to execute a monitoring application (not shown). The monitoring application may be executed as a stand-alone software application, as an application or may be accessible via a browser application (not shown).

In one or more embodiments, the workstation computer 284 is configured to: (i) receive data from the patient support apparatuses 210, the wireless locating nodes 230, and the server 240; and (ii) transmit data the patient support apparatuses 210, the wireless locating nodes 230, and the server 240. Optionally, in some embodiments, the workstation computer 284 may receive and/or transmit data to the mobile tags 220.

As a non-limiting example, the workstation computer 284 may receive and display, via monitoring application, data related to the subsystems and components of the patient support apparatus 210 (e.g., bed status, bed exit, alerts, etc.), the wireless mobile tags 220, the wireless locating nodes 230, the real-time location data and transmit control data to at least some of the subsystems and components of the patient support apparatus patient support apparatus 210, the wireless mobile tags 220, and the wireless locating nodes 230.

The system 200 comprises client devices 282 (only one instance shown in FIG. 2), associated with one or more users (not shown), such as medical personnel. As such, the client device 282 can sometimes be referred to as a “computing device”, “end user device” or “client computing device”.

The client device 282 may be a tablet used by one or more users, such as medical staff. It will be appreciated that the client device 282 may be implemented as a server, a desktop computer, a laptop, a smartphone, a tablet, and the like without departing from the scope of the present technology.

The one or more communication networks 290 may include one or more of wireless local area networks (WLANs), wired local area networks (LANs), personal area networks (PANs), nurse call systems, wide area networks (WANs), cellular networks, internet of things (IoT) networks, mesh networks, virtual private networks (VPNs), optical fiber networks, and digital health platforms.

How a given communication link 295 (not separately numbered) between the patient support apparatuses 210, the server 240, the workstation computer 284, and the client device 282 is implemented will depend inter alia on how each computing device is implemented. It will be appreciated that each given communication link 295 may be of a different type (e.g., wired or wireless) and may form or be connected to a different type of network of the one or more communication networks 290.

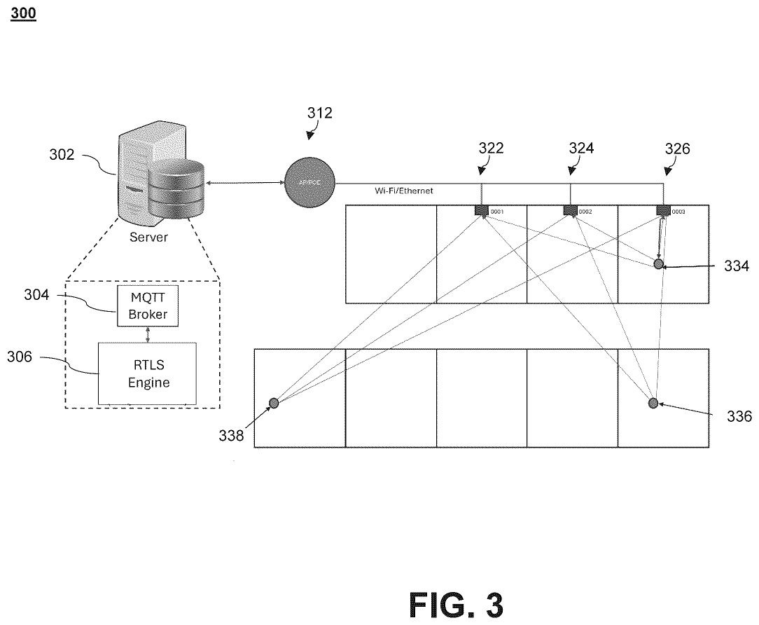

FIG. 3 illustrates a schematic diagram of an exemplary first environment 300 implementing a real-time locating system (RTLS) with mobile wireless tags in a medical facility in accordance with one or more non-limiting embodiments of the present disclosure.

In this embodiment, the first environment 300 comprises wireless locating nodes 322, 324, 326 with IR transceivers configured to confirm presence within a specific room, such as room 0003. The first environment 300 further comprises mobile tags 334, 336, 338, where each tag 334, 336, 338 includes a Bluetooth Low Energy (BLE) chip configured to broadcast wireless (e.g., Bluetooth iBeacon) signals, which are detectable by the plurality of wireless locating nodes 322, 324, 326 deployed throughout the facility.

Each room, including room 0003, and selected corridor zones are equipped with a wireless locating node 322, 324, 326 referred to herein as an “MQTT-Client” or “Anchor.” The wireless locating nodes 322, 324, 326 are connected to the local network infrastructure via Wi-Fi or Ethernet, which may include connectivity through access points or Power over Ethernet (PoE) switches 312.

The wireless locating nodes 322, 324, 326 are configured to receive BLE signals emitted by the tags 334, 336, 338. The infrared receiver in each wireless locating node 322, 324, 326 is further configured to detect presence specifically within rooms equipped with IR transmission beams, thereby identifying occupancy at a high level of spatial granularity.

The wireless locating nodes 322, 324, 326 are configured to transmit distance-related or proximity-related data, including Received Signal Strength Indicator (RSSI) values and IR detection status, to a central server 302 using the MQTT protocol. The central server 302 executes a real-time location system (RTLS) engine 306 that aggregates and processes data from all nodes.

With respect to position interpretation, the BLE-based iBeacon signals are analyzed by assessing variations in the received signal strength, which are used to characterize the distance between the tag and each node. The resulting proximity classification includes categories such as “Far,” “Close,” “Out,” or “In Room.” The wireless locating node 322, 324, 326 each transmit an infrared signal within the room; if the tag 334, 336, 338 receives this signal, the node registers the patient's presence within the specific room, such as room 0003. It will be appreciated that there may be two or more possible categories of classification.

Based on the analysis of BLE and IR data, the RTLS engine 306 executed on the server 302 determines the tag location. If the tag has received the IR signal and is concurrently detected as being within the room by BLE-based analysis, the RTLS engine 306 identifies the tag 334 as being located in room 0003. If the IR signal is not received but BLE proximity suggests spatial adjacency, the RTLS engine 306 may identify the tag 336 as being close to but outside room 0003 with a label such as, for example, “Close 0003 (Out).” If the RSSI indicates a more distant location, the tag 338 may be classified as being far from and outside room 0003 with a label such as, for example, “Far 0001 (Out).”

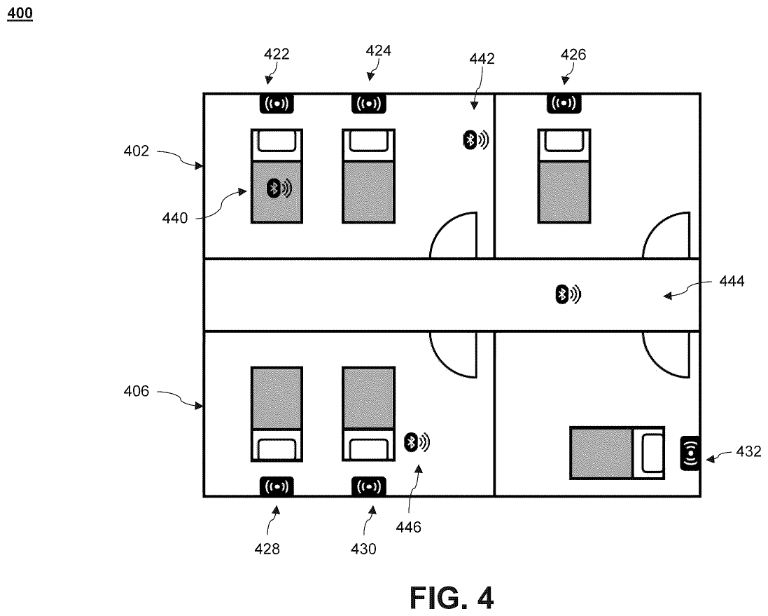

FIG. 4 illustrates a schematic diagram of a second environment 400 in a medical facility where different locations of mobile tags attached to patients or equipment are shown in accordance with one or more non-limiting embodiments of the present disclosure. The second environment 400 is a typical environment in which embodiments of the present technology may be implemented. In FIG. 4, there is one wireless node 422, 424, 426, 428, 430, 432 per clinical position, such that multiple wireless nodes may be present in the same room, such as for example shared room 402 including wireless nodes 422, and shared room 406 including wireless nodes 428, 430, where each wireless node is positioned to cover a zone including a bed efficiently. For purposes of localization of the mobile tags 440, 442, 444, 446, only one wireless node may be used per room, and nodes should be installed in zones where monitoring is desired, such as hospital wings, storage closets, hallways, etc. To optimize the localization of tags, nodes may be distributed as evenly as possible, with a constant distance between them to ensure optimal coverage.

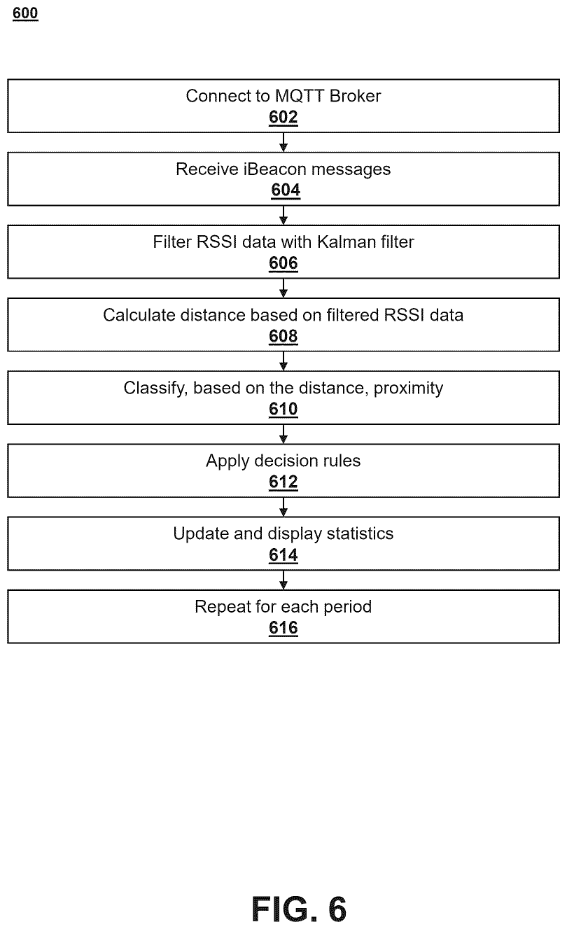

FIG. 6 is a flow chart of a first procedure 600 for locating mobile tags in accordance with one or more non-limiting embodiments of the present disclosure.

The first procedure 600 is executed by one or more computing devices including one or more processing units operatively connected to a non-transitory storage medium. In one or more embodiments, the computing device executing the first procedure 600 includes the server 240 of FIG. 2.

At processing step 602, the at least one processing unit connects to the MQTT Broker. The at least one processing unit establishes a connection with the MQTT broker to receive iBeacon messages from MQTT clients. In one or more embodiments, the MQTT broker is executed by the server 240 of FIG. 2.

At processing step 604, the at least one processing unit receives iBeacon messages. Messages from iBeacon tags are received via the MQTT channel, containing the necessary information (e.g., RSSI values). The iBeacon messages are messages received by the wireless nodes and broadcast by mobile tags. The iBeacon messages include wireless signal parameters detected by the wireless nodes. The wireless signal parameters include RSSI values.

At processing step 606, the at least one processing unit filters RSSI data with a Kalman Filter. The RSSI (Received Signal Strength Indicator) data values are filtered to reduce fluctuations and improve calculation accuracy. In alternative embodiments, other types of filters may be used instead of Kalman filters.

At processing step 608, the at least one processing unit calculates a distance based on the filtered RSSI data. The distances between the mobile tags and the nodes (i.e., anchors) are calculated using a signal loss model based on the RSSI data values.

At processing step 610, the at least one processing unit classifies proximity based on the distance. The nodes (anchors) are classified into proximity categories such as one of Very Close, Close, and Far based on the calculated distances.

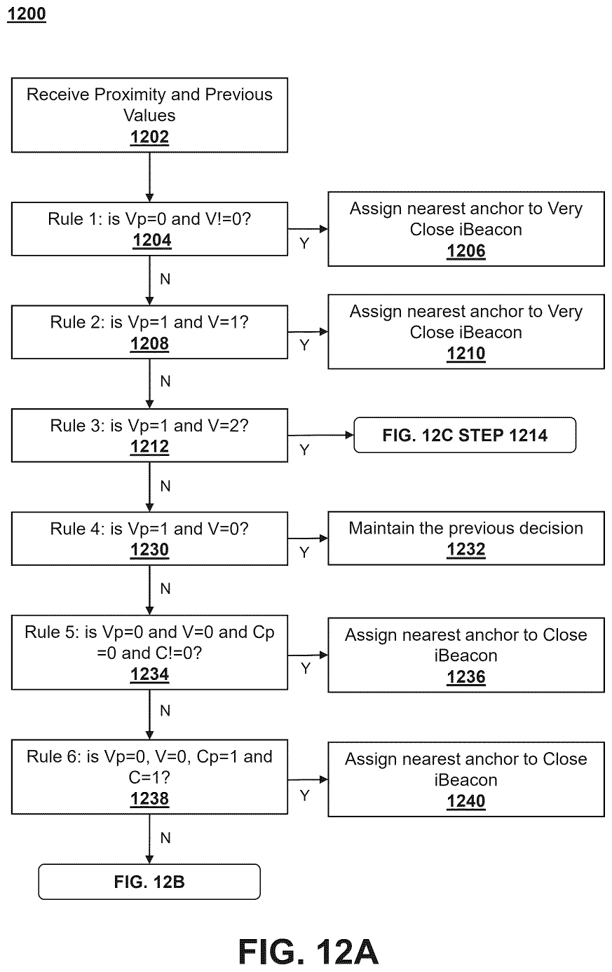

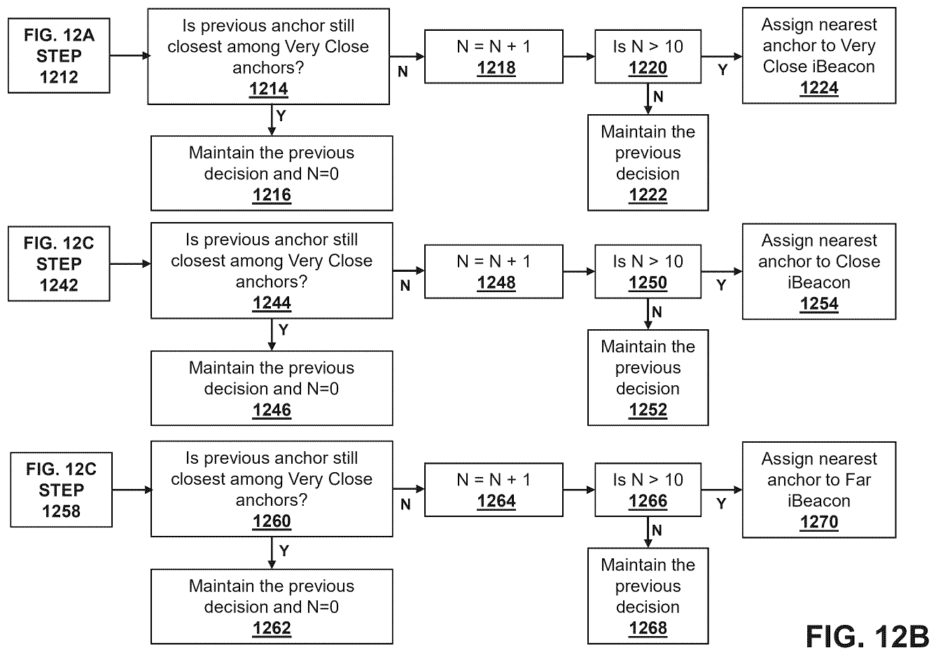

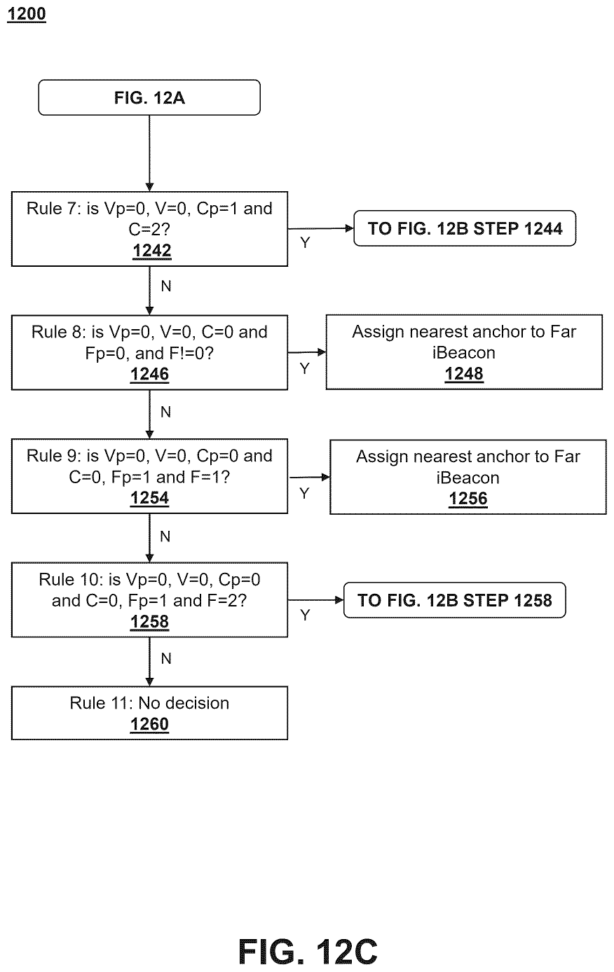

At processing step 612, the at least one processing unit applies decision rules. The decision rules are applied to determine which wireless node to associate with each iBeacon based on proximity data. Non-limiting examples of decision rules will be described hereinafter with reference to FIGS. 12A to 12C.

At processing step 614, the at least one processing unit updates and optionally causes display of statistics. The processed statistics and information are updated and displayed on the user interface, such as the nurse workstation computer 284 of FIG. 2.

At processing step 616, the at least one processing unit repeats the procedure 600 for each period for each configured iBeacon.

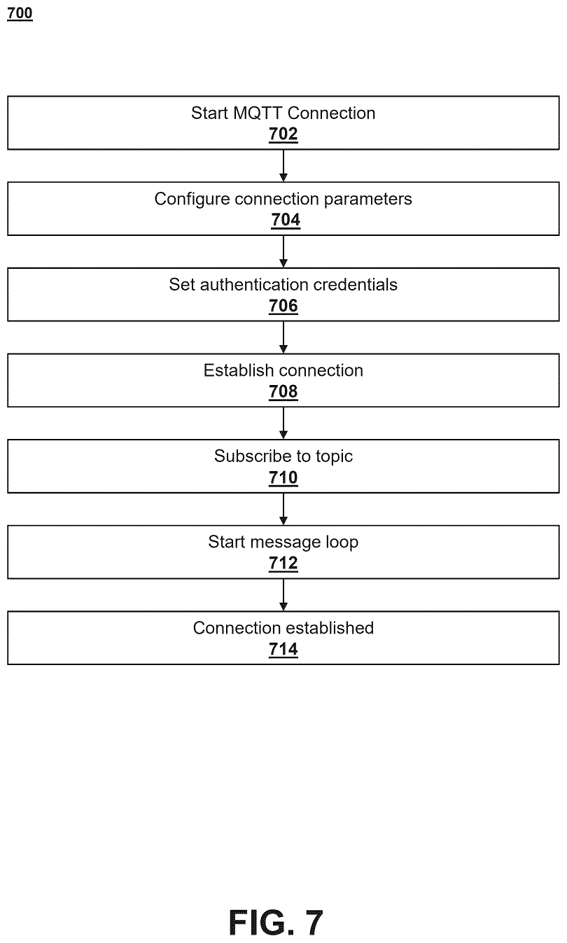

FIG. 7 is a flow chart of a procedure 700 for connecting to a Message Queuing Telemetry Transport (MQTT) broker in accordance with one or more non-limiting embodiments of the present disclosure.

In one or more embodiments, the MQTT broker may be the MQTT broker 258 executed by server 240 of FIG. 2. The procedure 700 is executed by at least one processing unit of a computing device, such as a processing unit of each of the plurality of wireless locating nodes 230 of FIG. 2.

At processing step 702, the at least one processing unit initiates the MQTT connection by preparing the necessary computing resources to establish a secure connection with the MQTT broker. The at least one processing unit launches the MQTT client and invokes the requisite functions to configure the client's connection settings.

At processing step 704, the at least one processing unit proceeds to configure the connection parameters, including the broker's address, the communication port, and the TLS certificate, to ensure the security of the communication channel through SSL/TLS encryption, which protects the data exchanged between the client, server and the MQTT broker.

At processing step 706, the at least one processing unit sets the authentication credentials, including the username and password, which are required by the MQTT broker. The server provides these credentials to the MQTT client via the username_pw_set function, thereby ensuring that only authorized clients can establish a connection with the broker.

At processing step 708, the at least one processing unit establishes the connection with the MQTT broker using the previously configured settings, including the broker address, port, and authentication information. Upon successful connection, the at least one processing unit is able to interact with the broker.

At processing step 710, the at least one processing unit subscribes to one or more predefined MQTT topics, enabling the at least one processing unit to receive messages published to these topics. The at least one processing unit subscribes to a specific topic using the subscribe method, thereby enabling the processing unit to receive relevant data, such as iBeacon information, published to the subscribed topic.

At processing step 712, the at least one processing unit initiates a message processing loop, wherein a thread is started to continuously listen for incoming messages from the MQTT broker. The at least one processing unit processes the received messages as they arrive in real-time, ensuring that it can interact with the broker and process the transmitted data without delay.

At processing step 714, the MQTT connection is fully established, and the at least one processing unit is capable of receiving and processing real-time messages from the broker, thereby enabling efficient and secure data exchange.

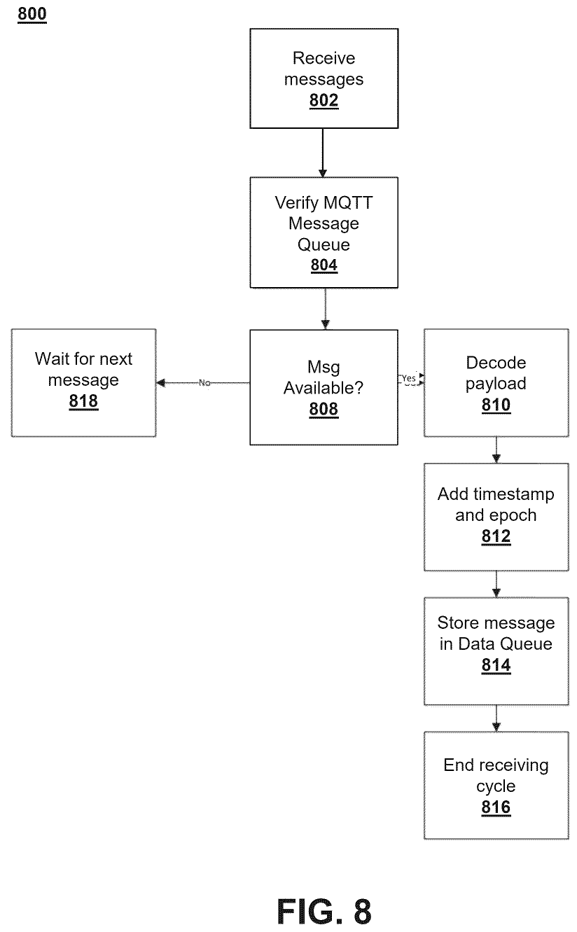

FIG. 8 is a flow chart of a third procedure 800 for receiving iBeacon messages in accordance with one or more non-limiting embodiments of the present disclosure.

The third procedure 800 for receiving iBeacon messages is executed by at least one processing unit, such as the processing unit of the server 240 of FIG. 2.

The at least one processing unit begins the third procedure 800 at processing step 802 by receiving messages by initiating the reception logic. The at least one processing unit activates a listening loop to monitor incoming messages, preparing the system to interact with the MQTT message queue. This step ensures that the system is prepared to handle messages from the MQTT broker as they arrive.

At processing step 804, the at least one processing unit verifies the MQTT message queue. The at least one processing unit determines if any messages are available for the topic to which the MQTT client is subscribed (e.g., /topic/ibeacon) at processing step 808. The at least one processing unit calls the appropriate function to extract messages from the queue, and if messages are available at processing step 810, the at least one processing unit proceeds to the next processing step 812. If no messages are available, the at least one processing unit continues to wait for new messages to arrive at processing step 814.

At processing step 808, the at least one processing unit verifies whether any messages have been extracted from the MQTT message queue. If messages are present, the at least one processing unit processes each message individually and proceeds to decode the JSON payload of the message at processing step 816. If no messages are available, the at least one processing unit moves to the “No Message Available” step, where it waits before rechecking the queue.

At processing step 810, upon receiving a message, the at least one processing unit decodes the JSON payload to extract the relevant data. This is achieved using a JSON parsing library, such as json.loads, to decode the message payload and prepare the data for further processing. If the JSON is malformed, an error message is generated, but the at least one processing unit continues to listen for new messages.

At processing step 812, the at least one processing unit enriches the decoded message by adding a timestamp and an epoch time to the data. The timestamp indicates the current date and time, while the epoch time represents the number of seconds since Jan. 1, 1970. This additional metadata enables precise tracking of when each message was received. The at least one processing unit uses Python's datetime and time libraries to add these fields to the message.

At processing step 814, the at least one processing unit stores the message in a data queue for further processing. The at least one processing unit uses a queue data structure, such as queue.Queue( ), to store the message in memory for processing by other parts of the system. This ensures the messages are organized and ready for subsequent handling.

If no messages are available in the MQTT queue, the at least one processing unit enters a waiting mode at processing step 818, pausing briefly before rechecking the queue. This avoids unnecessary resource consumption by allowing the at least one processing unit to wait for messages to arrive. Once a message is available, the process resumes automatically.

The at least one processing unit incorporates a small waiting period to synchronize the reception of messages. This pause, such as a time.sleep(1) call, allows messages to accumulate in the queue, optimizing system resource usage while ensuring adequate responsiveness.

At processing step 816, the at least one processing unit prepares for the next cycle of message reception. The at least one processing unit's listening loop continues, allowing the program to monitor the MQTT message queue and handle incoming messages in real-time. This ensures continuous operation and the receipt of new messages as they become available.

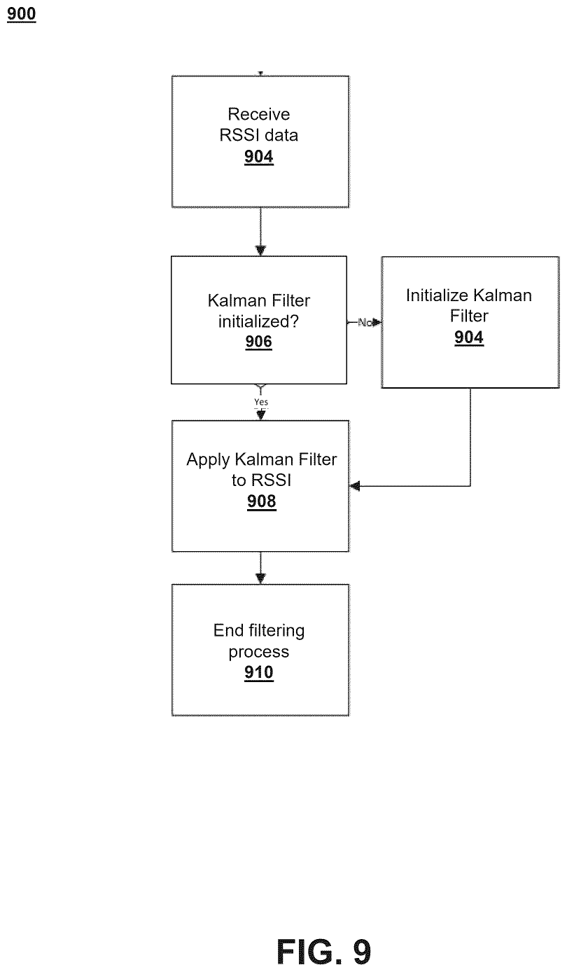

FIG. 9 is a flow chart of a fourth procedure 900 for filtering the RSSI values with a Kalman Filter in accordance with one or more non-limiting embodiments of the present disclosure.

The fourth procedure 900 is executed by at least one processing unit, such as the at least one processing unit of the server 240.

The purpose of the fourth procedure 900 is to smooth the fluctuations of the received signal strength indicator (RSSI) values to obtain more accurate data by removing noise from the RSSI measurements.

The at least one processing unit proceeds by receiving the raw RSSI data for each iBeacon mobile tag and wireless locating node at processing step 904. The RSSI data are extracted from the MQTT messages that have been received from the MQTT broker.

The at least one processing unit then verifies whether a Kalman filter has already been initialized for the specific iBeacon mobile tag and wireless node at processing step 906. If the Kalman filter already exists, the at least one processing unit proceeds directly to applying the Kalman filter to the RSSI data at processing step 908.

If no filter exists, the at least one processing unit initializes a new Kalman filter for the corresponding iBeacon mobile tag and wireless node at processing step 904.

The initialization of the Kalman filter involves setting several parameters, including: process noise (q), measurement noise (r), estimation error (p), and the initial state estimate (x). The at least one processing unit sets the following values for the filter:

Process Noise (q) reflects the expected variability in the underlying data.

Measurement Noise (r) accounts for the uncertainty in the measured RSSI values.

Estimation Error (p) is the initial estimation error.

The Initial Estimate (x) is set to 0.

The Kalman filter is initialized using the equations:

x₀=0,p₀=estimationₑᵣᵣₒᵣ,q=processₙₒᵢₛₑ,r=measurementₙₒᵢₛₑ

Once the Kalman filter is initialized, the at least one processing unit applies the filter to the current RSSI values to obtain a filtered estimate. This procedure involves two steps:

Prediction Step: The estimation error is updated using the equation:

$${p}_{k}^{-} = {p}_{k - 1} + q \\ where \\ {p}_{k}^{-}$$

is the a priori estimation error, pₖ₋₁ is the previous estimation error, and q is the process noise.

### Measurement Update Step:

The Kalman gain is computed using equation:

$${K}_{k} = \frac{{p}_{k}^{-}}{{p}_{k}^{-} + r}$$

where Kₖ is the Kalman gain and r is the measurement noise.

The state estimate is then updated using equation:

xₖ=xₖ₋₁+Kₖ·(zₖ−xₖ₋₁).
where xₖ is the updated estimate, zₖ is the current RSSI measurement, and xₖ₋₁ is the previous estimate.

Finally, the estimation error is updated using equation:

$${K}_{k} = \frac{{p}_{k}^{-}}{{p}_{k}^{-} + r}$$

As a result of this filtering process, the at least one processing unit obtains a filtered RSSI value, denoted as (xₖ), which reduces the impact of noise in the RSSI measurements.

The filtered RSSI value is then stored for further processing, such as calculating the distance between the iBeacon mobile tag and the wireless node. This ensures that the filtered data is available for subsequent stages of the system.

The filtering process concludes once all RSSI measurements for the iBeacon mobile tag and wireless node have been processed. At this point, at processing step 910 the at least one processing unit completes the filtering cycle and is ready to begin the filtering process for the next set of measurements.

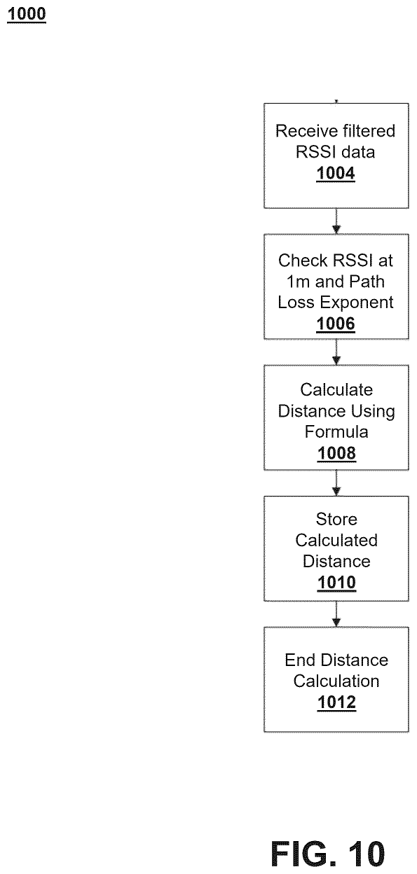

FIG. 10 is a flow chart of a fifth procedure 1000 for calculating an estimated distance based on the RSSI in accordance with one or more non-limiting embodiments of the present disclosure.

The fifth procedure 1000 is executed by at least one processing unit. In one or more embodiments, the fifth procedure 100 is executed by the at least one processing unit of the server 240.

The purpose of the fifth procedure 1000 is to apply the logic to convert the filtered RSSI values into an estimated distance, where data for each wireless node and iBeacon mobile tag is used to estimate the distance based on the received signal strength indicator values.

The at least one processing unit receives the filtered RSSJ data at processing step 1002, which has previously been processed by the Kalman filter. The at least one processing unit extracts the filtered RSSI values for each wireless node from the corresponding data structure, such as a DataFrame structure. This ensures that the distance calculations are based on reliable and smoothed RSSI values, which contribute to the accuracy of the distance estimation.

Before calculating the distance, the at least one processing unit verifies two parameters at processing step 1004:

RSSI at 1 m: The RSSI value measured at a distance of 1 meter, which serves as a constant parameter defined for each environment or device.

Path Loss Exponent (PLE): A constant that reflects the attenuation of the signal in the environment, such as walls or other obstacles.

The at least one processing unit retrieves these parameters from the configuration or available data. It will be appreciated that distance calculation is directly influenced by these parameters, as they account for the initial signal strength and environmental factors that affect the signal propagation.

At processing step 1006, the at least one processing unit calculates the distance using a simplified logarithmic propagation model. The formula for distance calculation is as follows:

$$d = 10 \frac{{RSSI}_{@ 1 m} - {RSSI}_{filtred}}{10 \cdot n}$$

Where:
d is the estimated distance in meters,
RSSI_{@1m} is the signal strength at a distance of 1 meter,
RSSI_{filtered}: is the RSSI value after Kalman filtering,
n is the path loss exponent, which varies depending on the environment.

Once the distance is calculated, the at least one processing unit stores the calculated distance for each anchor associated with an iBeacon at processing step 1010. The calculated distances are added to the data structure, such as a DataFrame, for further processing. These stored distances are used for subsequent stages, such as proximity classification in two or more classes (e.g., “Close” and “Far”; or “Very Close,” “Close,” and “Far”).

The distance calculation procedure concludes at processing step 1012 once all distances for the available RSSI data have been calculated.

The at least one processing unit then transitions to the next step in the process, such as categorizing the distances or displaying the results to the user.

The procedure 1000 ends.

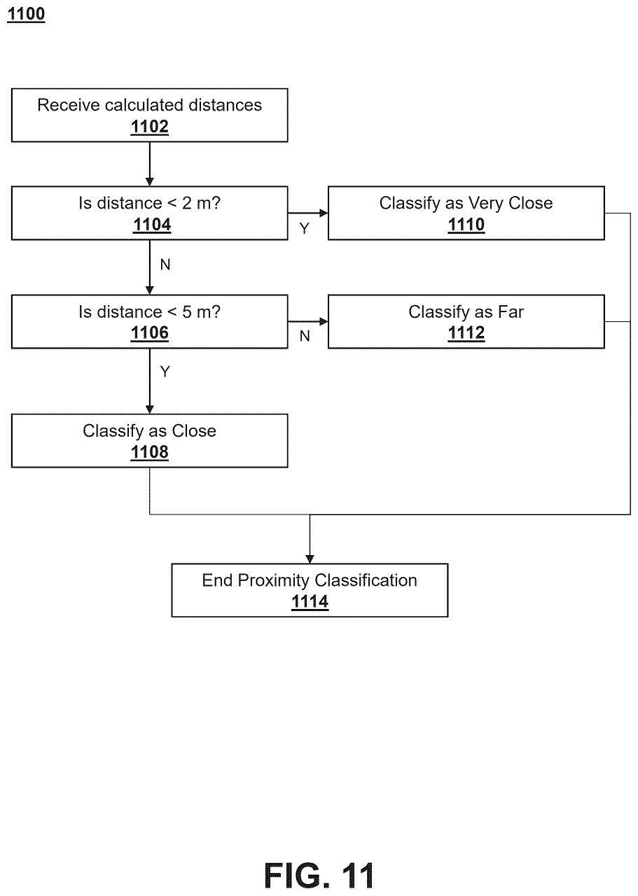

FIG. 11 is a flow chart of a procedure 1100 for proximity classification based on the estimated distance in accordance with one or more non-limiting embodiments of the present disclosure.

In one or more embodiments, the procedure 1100 is executed by at least one processing unit, such as the at least one processing unit of the server 240 implementing the RTLS engine 252.

The purpose of the proximity classification procedure 1100 is to classify the previously calculated distances based on predefined thresholds. The proximity classification procedure 1100 applies the logic to analyze the distances and translate them into interpretable proximity categories.

The at least one processing unit prepares the calculated distances for classification according to these thresholds, ensuring that the data is organized and ready for analysis.

At processing step 1102, the at least one processing unit receives the calculated distances, which were previously obtained from the filtered RSSI data by executing procedure 1000. These distances are associated with specific wireless nodes and iBeacon mobile tags. The at least one processing unit accesses the stored distances from the data structure, such as a DataFrame. This ensures that only the relevant data is used for classification, maintaining the accuracy and consistency of the classification process.

At the next processing step 1104, the at least one processing unit verifies if the distance is less than 2 meters, a threshold used to indicate very close proximity. The at least one processing unit compares the calculated distances with the 2-meter threshold.

If the distance is less than 2 meters, the at least one processing unit classifies the iBeacon mobile tag as being “Very Close” at processing step 1110. The at least one processing unit then adds the proximity label “Very Close” to the corresponding distance in the data structure. If the distance is greater than or equal to a first threshold distance (e.g., first threshold distance of 2 meters), the at least one processing unit moves to the next processing step 1106.

For distances that are not less than 2 meters, the at least one processing unit verifies if the distance is less than a second threshold distance (e.g., second threshold distance of 5 meters) at processing step 1106, which is considered a reasonable proximity but not immediate. The at least one processing unit compares the distance with the second threshold distance (e.g., meters), and if the distance is less than 5 meters but greater than or equal to 2 meters, it classifies the iBeacon as “Close” at processing step 1108. The proximity label “Close” is then added to the corresponding distance in the data structure. If the distance exceeds 5 meters, the at least one processing unit proceeds to the next classification step 1112.

For distances greater than or equal to 5 meters, the at least one processing unit classifies the iBeacon as “Far” at processing step 1112. The proximity label “Far” is then added to the corresponding distance in the data structure. This step ensures that all distances greater than 5 meters are categorized as “Far.”

At processing step 1114, once all distances have been classified, the at least one processing unit terminates the proximity classification procedure 1100.

The data structure now contains the proximity labels for each distance, which can be used for further analysis or for display in the user interface. The at least one processing unit ensures that the proximity classification is complete and ready for subsequent stages of the system's operation.

TABLE 1 provides an example of four terminals identified via MAC address which have been classified in terms of proximity according to their estimated distances to a mobile tag:

| Terminal MAC ID | Distance | Proximity |
|---|---|---|
| 17:00:00:00:00:00 | 0.5623 | Very Close |
| 1D:00:00:00:00:00 | 0.8796 | Very Close |
| 2A:00:00:00:00:00 | 3.8796 | Close |
| C1:00:00:00:00:00 | 5.6234 | Far |

FIGS. 12A, 12B and 12C illustrate a flow chart of a procedure 1200 for applying decision rules in accordance with one or more non-limiting embodiments of the present disclosure.

The procedure 1200 for applying decision rules is executed by at least one processing unit, such as the at least one processing unit of the server 240 (FIG. 2). It will be appreciated that the server 240 may receive data required for executing the procedure 1200 from the set of wireless locating nodes 230 (i.e., terminals) and/or the mobile tags 220 either directly or indirectly. The purpose of the procedure 1200 is to analyze the current proximity values and the historical proximity values to apply the relevant decision rules for associating a given wireless node to a mobile tag.

The procedure 1200 begins at processing step 1202, where the at least one processing unit receives data and extracts the current proximity values (Very Close (V), Close (C), Far (F)) where: 0 corresponds to no wireless node detected; 1 corresponds to one wireless node detected; and 2 corresponds to a plurality of wireless nodes detected). The procedure 1200 extracts the historical proximity values (Vp, Cp, Fp) where: 0 corresponds to 0 wireless nodes detected historically; and 1 corresponds to at least one wireless node detected historically.

At processing step 1204, Rule 1 is triggered when Vp=0 et V=1; which corresponds to the transition from historical “Very Close” (Vp=0) to “Very Close” (V=1).

If this condition is met, the wireless node is immediately assigned to the Very Close category at processing step 1206.

If this condition is not satisfied, the procedure 1200 proceeds to Rule 2 at processing step 1208.

Rule 2 is triggered when Vp=1 and V=1; which corresponds to the wireless node remaining in Very Close (Vp=1, V=1) proximity (i.e., no change from historical to current proximity). If this is the case, at processing step 1210, the wireless node remains in the Very Close category for proximity to the tag.

If not, the procedure 1200 proceeds to Rule 3 at processing step 1212.

Rule 3 is triggered when the procedure 1200 detects current multiple wireless nodes in Very Close proximity (V=2) after having detected only one in Very Close previously (Vp=1). In this case, the procedure 1200 verifies at processing step 1214 if the previously selected wireless node remains the closest in Very Close proximity.

If so, the previous decision is maintained at processing step 1216 (the wireless node corresponding to very close is maintained); if not, at processing step 1218, a counter N is incremented (N=N+1). If N exceeds a threshold (e.g., 10), at processing step 1220, the previous decision is retained (processing step 1222), but if N is still less than the threshold (e.g., 10), the procedure 1200 assigns the closest wireless node to the Very Close proximity category at processing step 1224.

Rule 4 is triggered at processing step 1230 when the procedure 1200 detects a wireless node historically being in Very Close proximity (Vp=1) and detects no wireless nodes currently in Very Close (V=0) proximity.

In this case, at processing step 1232, the previous decision is retained where the current wireless node is kept, and no change is made.

If not, the procedure 1200 proceeds to Rule 5 at processing step 1234.

Rule 5 is triggered when: the procedure 1200 detects that there was no previous detection of wireless nodes in Very Close proximity (Vp=0, V=0) and the procedure 1200 did not detect wireless nodes in historical close proximity (Cp=0) and currently detects at least one wireless node in Close proximity (C≠0).

If this condition holds, at processing step 1236, the closest wireless node is assigned to the Close proximity category.

If not, the process proceeds to Rule 6.

Rule 6 is triggered at processing step 1238 when: the procedure 1200 determines no historical and no current wireless nodes in very close proximity (Vp=0, V=0) and detects a wireless node that was historically in close proximity that currently remains in close proximity (Cp=1, C=1).

If so, the procedure 1200 resumes with the current Close proximity assignment of the wireless node at processing step 1240.

Rule 7 is triggered at processing step 1242 when the procedure 1200 determines that multiple wireless nodes are in Close proximity after historically in Close proximity (Cp=1, C=2), which corresponds to (Vp=0, V=0, Cp=1, C=2)

The procedure 1200 verifies if the previously selected terminal remains the closest at processing step 1244, and if so, maintains the decision, resetting the counter N to 0 at processing step 1246. If the closest terminal has changed, the procedure 1200 increments N at processing step 1248 and verifies whether N exceeds 10 at processing step 1250. If it does, the previous decision is retained at processing step 1252; otherwise, the closest terminal is reassigned to the Close category at processing step 1254.

Rule 8 is triggered at processing step 1246 when the procedure 1200 detects at least one terminal currently in the Far category after not being historically in either Very Close or Close proximity previously, which corresponds to (Vp=0, V=0; Cp=0, C=0; F≠0).

If this condition holds, the closest terminal is assigned to the Far category at processing step 1248. If the conditions does not hold, the procedure 1200 proceeds to Rule 9.

Rule 9 is triggered at processing step 1254 when the procedure 1200 determines that and a terminal was previously in Far proximity (Fp=1) and currently remains in Far proximity (F=1), which corresponds to (Vp=0, V=0, Cp=0, C=0, Fp=1, F=1). The procedure 1200 continues with the current Far proximity assignment.

Rule 10 is triggered at processing step 1258 when the procedure 1200 determines that a terminal was previously in Far proximity (Fp=1) and currently determines that multiple terminals are in Far proximity (F=2), which corresponds to (Vp=0, V=0, Cp=1, C=0, Fp=1, F=2)

If the previously selected terminal remains the closest, the procedure 1200 maintains the decision at processing step 1262, resetting N to 0. If the closest terminal has changed, the counter N is incremented (N=N+1) at processing step 1264 and the threshold condition is verified (N>10) at processing step 1266. If N exceeds 10, the previous decision is kept at processing step 1268, but if not, the closest terminal is reassigned to the Far proximity category at processing step 1270.

Rule 11 handles any incoherent or undefined situation where none of the previous categories match at processing step 1260. In such cases, no decision is made, and the procedure 1200 leaves the terminal assignment undefined. This decision process ensures that the terminal assignment is always based on the most appropriate and consistent proximity data, with necessary safeguards in place for transitioning between proximity categories.

The procedure 1200 then ends.

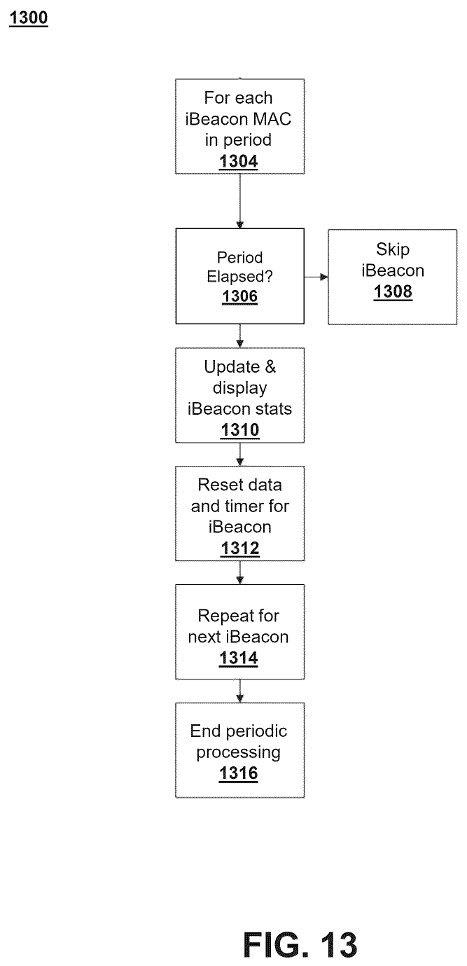

FIG. 13 is a flow chart of a periodic processing 1300 for mobile tags in accordance with one or more non-limiting embodiments of the present disclosure.

The periodic processing 1300 is executed by at least one processing unit, such as the processing unit of the server 240.

The process of periodic processing 1300 begins with the initiation of the periodic processing loop for the iBeacons. This step prepares the loop that will iterate over each configured iBeacon. The at least one processing unit starts by loading the list of iBeacons defined in IBEACON_PERIODS and initializing the necessary variables to track periods and associated data for each iBeacon.

A loop is then established to process each iBeacon that has a defined period at processing step 1304. For each iteration, the at least one processing unit extracts the MAC address of the current iBeacon and retrieves the period assigned to it from IBEACON_PERIODS. This allows the at least one processing unit to handle each iBeacon individually according to its specific configuration.

The at least one processing unit then verifies at processing step 1306 whether the time elapsed since the last update for this iBeacon exceeds the configured period. This ensures that data is processed at regular intervals. If the elapsed time is greater than the period specified in IBEACON_PERIODS, the at least one processing unit proceeds to the next step. If the period has not elapsed, the at least one processing unit moves on to the next iBeacon without performing any further processing.

If no data is available or if the period has not yet elapsed, the iBeacon is ignored for this cycle at processing step 1308. A message is displayed indicating that no data is available or that the period has not yet been reached, thus avoiding unnecessary processing.

If the period has elapsed, the at least one processing unit proceeds to update and display statistics for the current iBeacon at processing step 1310. This includes applying the Kalman filter to the RSSI data to smooth out fluctuations, calculating statistics such as the average RSSI, and computing distances based on the path loss model. The at least one processing unit then categorizes the terminals into proximity categories (Very Close, Close, Far) based on the calculated distances.

Once the statistics are updated, the accumulated data for the current iBeacon is reset, and the timer is updated for the next cycle. This ensures that the iBeacon is ready for the next processing cycle. Temporary data associated with the iBeacon is cleared, and the current time is recorded as the starting point for the next period.

After processing or ignoring the current iBeacon, the at least one processing unit moves on to the next iBeacon in the list at processing step 1314, repeating the steps for each one. This ensures that all configured iBeacons are processed in sequence.

Once all configured iBeacons have been processed or ignored, the periodic processing cycle ends at processing step 1316. The main program loop continues its execution, and data is prepared to be collected or displayed during the next cycle. The at least one processing unit is now ready to process the next interval of data.

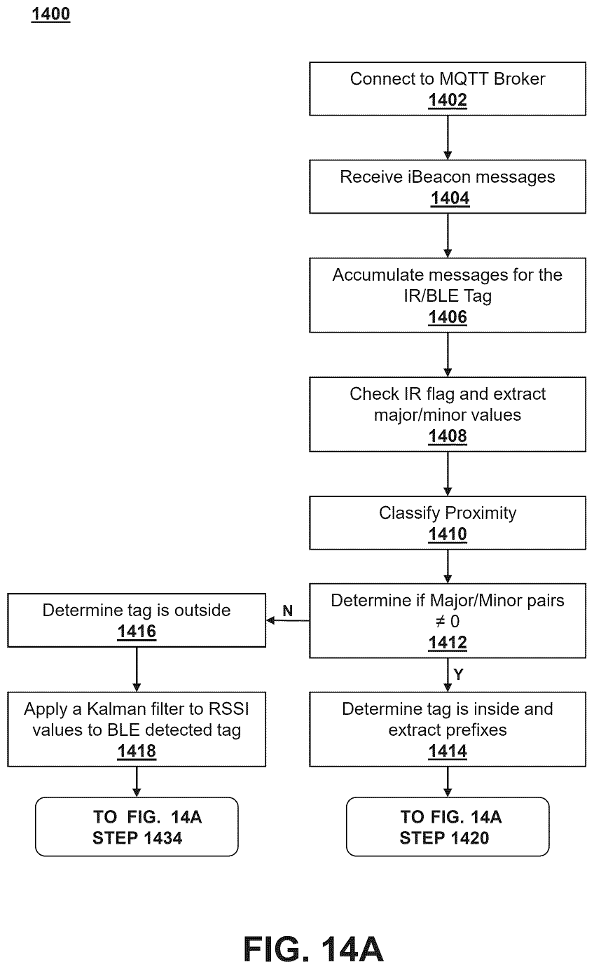

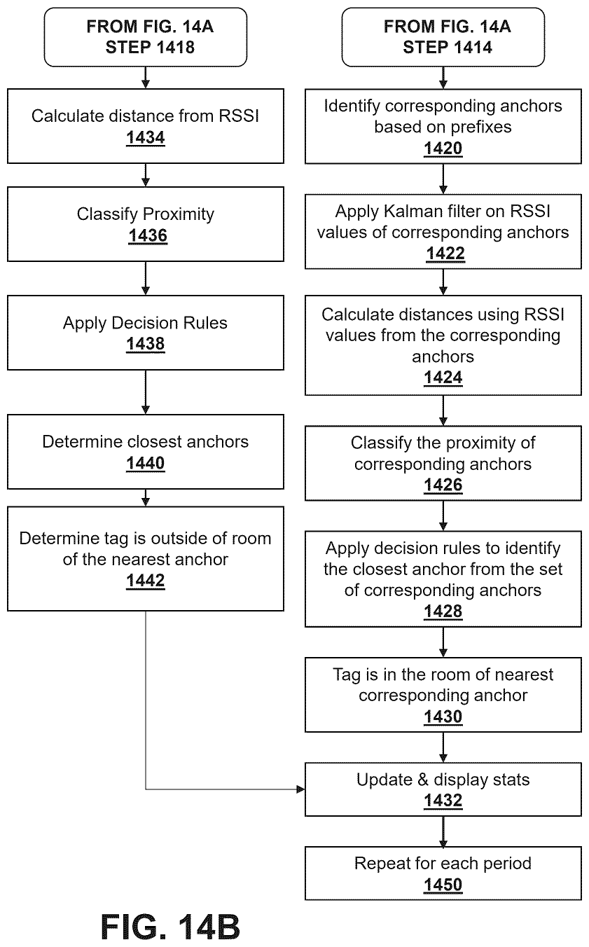

With reference to FIG. 14A and FIG. 14B, there is shown a flow chart of a procedure 1400 for locating mobile tags using infrared and Bluetooth signals in accordance with one or more non-limiting embodiments of the present disclosure.

The procedure 1400 may be executed by a RTLS engine, such as the RTLS engine 252 of FIG. 2. It will be appreciated that the RTLS engine 252 is executed by a computing device comprising at least one processing unit.

At processing step 1402, the at least one processing unit initiates operation by establishing a connection with a Message Queuing Telemetry Transport (MQTT) broker.

At processing step 1404, the at least one processing unit receives iBeacon messages transmitted by the IR/BLE mobile tag.

At processing step 1406, the at least one processing unit accumulates the received iBeacon messages associated with the IR/BLE mobile tag.

At processing step 1408, the at least one processing unit examines the infrared (IR) flag contained within the accumulated messages and extracts the corresponding Major and Minor values.

At processing step 1410, the at least one processing unit classifies the proximity of the tag based on the extracted values into one of several predefined proximity categories, including “Very Close,” “Close,” and “Far.”. It will be appreciated that alternative proximity categories may be used.

At processing step 1412, the at least one processing unit determines whether the Major and Minor value pairs are nonzero. If the major and minor value pairs are equal to zero, the procedure 1400 goes to step 1414. If the major and minor value pairs are not equal to zero, the procedure 1400 goes to step 1416

At processing step 1414, the at least one processing unit determines that the tag is located within the monitored environment and extracts prefixes from the previously obtained Major and Minor values.

At processing step 1420, the at least one processing unit identifies a set of corresponding terminals (i.e., wireless locating nodes) using the extracted prefixes as identifiers.

At processing step 1422, the at least one processing unit applies a Kalman filter to the Received Signal Strength Indicator (RSSI) values associated with the identified corresponding terminals.

At the next processing step, the at least one processing unit

At the next processing step 1424, the at least one processing unit calculates the distance between the tag and each of the corresponding terminals using the filtered RSSI values.

At the next processing step 1426, the at least one processing unit classifies the proximity of each of the corresponding terminals into the predefined proximity categories of “Very Close,” “Close,” and “Far.”

At the next processing step 1428, the at least one processing unit applies decision rules to determine which of the corresponding terminals is closest to the tag based on the classified proximity data.

At the next processing step 1430, the at least one processing unit establishes that the tag is located in the room corresponding to the nearest terminal within the identified set.

At processing step 1416, if the Major and Minor value pairs are determined to be zero, the at least one processing unit designates the tag as being outside the monitored environment.

At the next processing step 1418, the at least one processing unit applies a Kalman filter to the RSSI values as measured by all terminals that detected the tag's BLE transmissions.

At the next processing step 1434, the at least one processing unit calculates the distance between the tag and each terminal that detected it, using the measured RSSI values.

At the next processing step 1436, the at least one processing unit classifies the proximity of each detecting terminal into categories including “Very Close,” “Close,” and “Far.”

At the next processing step 1438, the at least one processing unit applies decision rules to the classified proximity data based on the calculated distances.

At the next processing step 1510, the at least one processing unit determines the terminal in closest proximity to the tag and concludes that the tag is located outside the room associated with the nearest terminal.

At processing step 1432, the at least one processing unit updates internal statistics and displays the resulting data.

At processing step 1450, the at least one processing unit repeats the entire process for each operational period.

The procedure 1400 then ends.

### Machine Learning Training and Inference Procedure

The following describes a training procedure and an inference procedure for a machine learning (ML) model to predict presence of a mobile wireless tag within a room. The training procedure may be executed for a set of ML models, where each model is associated with a respective locating node in a room.

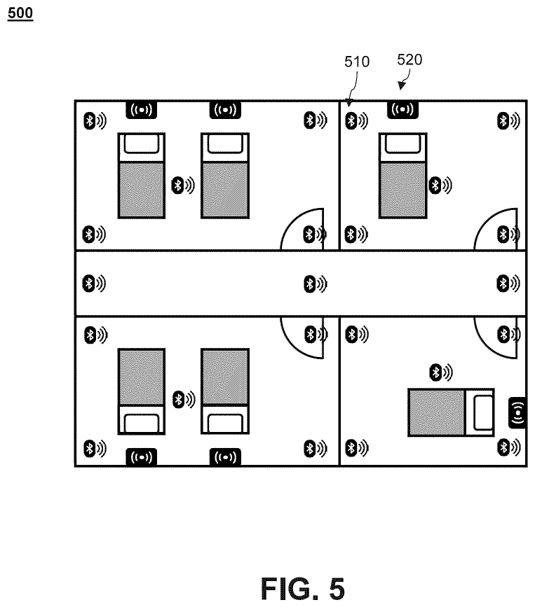

The acquisition and training procedure is configured to retrieve raw data and associate it with contextual information, including room number and position within the room. For each measurement point selected by operators, one or more mobile tags (e.g., iBeacons tags) are attached to a measurement rod, the measurement rod being a stick designed to hold the iBeacons at a height corresponding approximately to a height of the tag from the floor when worn by a patient or fixed to medical equipment (e.g., one meter from the floor). FIG. 5 shows a non-limiting example of an environment 500 in a medical facility where mobile tags 510 (only one numbered in FIG. 5) are positioned at different locations in each room and emit signals that are detected by the wireless locating nodes 520 (only one numbered in FIG. 5) and used for generating training data. In the example shown in FIG. 5, the mobile tags are distributed in the corners and center of each room.

Within the application, the room name and acquisition time are selected, after which a measurement can be conducted. Upon completion of all measurements, the raw data is stored. Table 2 provides an example of data following acquisition, it being noted that the actual table includes additional information beyond that presented, with certain data omitted to simplify the presentation.

TABLE 2
RSSI data for different wireless locating nodes and mobile tags

| Terminal MAC | iBeacon MAC | RSSI | Room | Position |
|---|---|---|---|---|
| . . . | . . . | . . . | . . . | . . . |
| 17:00:00:00:00:00 | C3:00:00:34:F5:00 | −42 | A | 3 |
| 17:00:00:00:00:00 | C3:00:00:34:F5:00 | −44 | A | 3 |
| 1D:00:00:00:00:00 | C3:00:00:34:F5:00 | −57 | A | 3 |
| 2A:00:00:00:00:00 | C3:00:00:34:F5:00 | −58 | A | 3 |
| 2A:00:00:00:00:00 | C3:00:00:34:F5:00 | −60 | A | 3 |
| C1:00:00:00:00:00 | C3:00:00:34:F5:00 | −69 | A | 3 |
| . . . | . . . | . . . | . . . | . . . |

Rows containing RSSI values lower than −100 are removed. The table is then pivoted to obtain a list representing the average RSSI observed by each access point for each combination of iBeacon MAC address, room number, and point. Table 3 presents the pivoted data derived from Table 2.

TABLE 3
pivoted table with RSSI data for different mobile tags

| iBeacon MAC | Room | Position | RSSI 17 | RSSI 1D | RSSI 2A | RSSI C1 |
|---|---|---|---|---|---|---|
| . . . | . . . | . . . | . . . | . . . | . . . | . . . |
| C3:00:00:34:F5:00 | A | 3 | −43 | −57 | −59 | −69 |
| . . . | . . . | . . . | . . . | . . . | . . . | . . . |
| C3:00:00:34:F5:00 | B | 2 | −70 | −33 | −39 | −43 |
| . . . | . . . | . . . | . . . | . . . | . . . | . . . |

When data is missing in the pivoted table, particularly when the acquisition time is short, which may also occur when at a certain position, an access point has not detected the iBeacon signal, in which case the missing data are filled with a predetermined or default value (e.g., −100). Furthermore, to increase the robustness of the system, data augmentation may be performed by duplicating the existing data and modifying the RSSI value of one access point per row to −100, then adding the modified data to the original data. In this manner, during prediction, if data are missing, they can be set to the default value of −100, which the neural network can interpret as a disconnected access point. Subsequently, a column indicating whether the data represent an iBeacon located inside the room is added. This step depends on the room under study and is repeated for each room. For example, when room A is under study, data originating from this room are marked as IN, whereas data originating from other rooms are marked as OUT, as shown in Table 4.

TABLE 4
IN/OUT data for mobile tags in room A

| iBeacon MAC | Room | Position | RSSIs | IN/OUT |
|---|---|---|---|---|
| C3:00:00:34:F5:00 | A | 3 | . . . | IN |
| C3:00:00:34:F5:00 | B | 2 | . . . | OUT |

The rows are then randomly shuffled before being separated into features and labels. Subsequently, twenty percent of the data are used to create a test set intended for evaluating the performance of the model. Prior to training the model, standardized preprocessing of the data is performed. A StandardScaler is applied to normalize the features, transforming the RSSI values so that they exhibit a zero mean and unit standard deviation. Concurrently, a LabelEncoder is employed to convert the categorical labels, IN and OUT, into binary numerical values, 1 and 0, thereby preparing the data for use with the machine learning algorithm. This standardization process ensures that all features contribute uniformly to the training of the model without any feature being overweighted due to its initial scale.

The neural network model is subsequently created according to the following architecture: four input neurons, one for each feature; two hidden layers comprising sixty-four and thirty-two neurons respectively, each employing a rectified linear unit (ReLU) activation function; and one output neuron with a sigmoid activation function, suitable for binary classification between IN and OUT. The model is compiled with the following parameters: optimizer set to Adam; loss function defined as Binary Cross-entropy; and performance evaluation carried out according to the accuracy metric. Training is conducted using the training set, and the model is then tested on the test set. At this stage, a list of models is generated, with one model corresponding to each room in the facility. The features, denoted as X, represent the data forming the input vector of the model, consisting of the RSSI values observed by each access point. The labels, denoted as y, correspond to the responses used during training, which the model will later predict, consisting of the IN or OUT classes.

In order to generate predictions for an iBeacon, the data are formatted in the same manner as during training. The order of the features in the pivoted table is maintained identical to the order used during training. The number of features corresponds exactly to that of the original dataset. The RSSI data are normalized using the same StandardScaler applied during training. Missing values are handled in the same manner, by being replaced with the value −100. For each model in the list, a probability of presence of the iBeacon in the room is generated, as shown in Table 5. The maximum probability is then evaluated. If the probability is greater than 0.5, the iBeacon is considered to be present in the room, denoted as IN. Otherwise, the iBeacon is considered to be absent from any room, denoted as OUT.

Table 5 presents the initial data converted into binary values, where IN represents the presence of the iBeacon in the room and OUT represents its absence.

| iBeacon | Room A | Room B | Room C | Room D |
|---|---|---|---|---|
| C3:00:00:34:F5:00 | 0.9917 | 0.6532 | 0.2356 | 0.1234 |
| C3:00:00:34:F5:01 | 0.1050 | 0.3095 | 0.1057 | 0.4999 |

In one or more alternative embodiments of the ML training procedure, iBeacon tags may be fixed to a set of positions in the room (e.g., each corner), and the signal parameters may be analyzed periodically (e.g., 24 h) to generate training data for determining the presence of the wireless tag in the room.

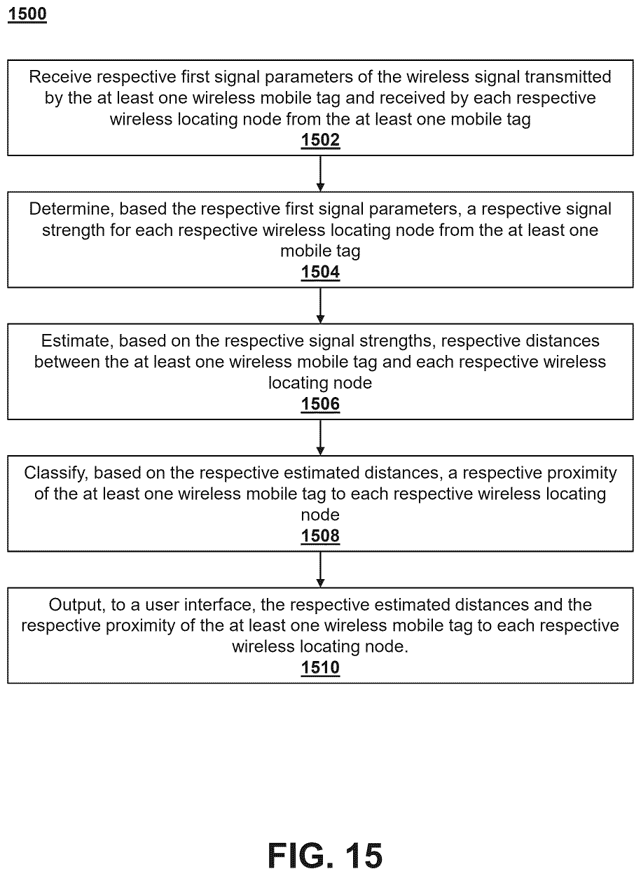

With reference to FIG. 15, there is illustrated a flowchart of a method 1500 of locating wireless mobile tags in accordance with one or more non-limiting embodiments of the present technology. The method 1500 may be executed within the environment of FIG. 2.

In one or more embodiments, the method 1500 is executed by at least one processing unit. The at least one processing unit is operatively connected to a non-transitory storage medium storing computer-readable instruction thereon. The at least one processing unit, upon executing the computer-readable instructions, is configured to or operable to execute method 1500.

In one or more embodiments, the method 1500 is executed by the RTLS engine 252 executed by the server 240. The server 240 is operatively connected to the plurality of wireless nodes 230 over the communication network 290. The plurality of wireless nodes 230 are configured for receiving wireless signals transmitted by the plurality of wireless tags 220.

The method 1500 begins at processing step 1502.

At processing step 1502, the at least one processing unit receives respective first signal parameters of the wireless signals transmitted by the at least one wireless mobile tag and received by each respective wireless locating node from the at least one mobile tag.

Each of the plurality of wireless tags 220 transmits wireless signals, which are detected by the wireless locating nodes 230 in range. Each wireless locating node 230 in range measures first wireless signal parameters of the wireless signals received by nodes in range.

In one or more embodiments, each of the plurality of wireless tags 220 includes a respective Bluetooth beacon and is associated with an identifier.

In one or more embodiments, the first wireless signal parameters may include one or more of power-based parameters (RSSI, RSRP, path-loss, including received power metric, carrier frequency and channel number, antenna gain/pattern descriptors, receiver gain, environment model terms). As a non-limiting example, the wireless signal may be a Bluetooth Low Energy signal, and the wireless signal parameters include at least the RSSI and a unique identifier of the tag.

At processing step 1504, the at least one processing unit determines, based the respective first signal parameters, a respective signal strength for each respective wireless locating node from the at least one mobile tag.

In embodiments using RSSI, the RTLS engine 252 determines the signal strength at each wireless node of the plurality of wireless nodes 230 with respect to each wireless tag of the plurality of wireless tags 220 in range. The RTLS engine 252 applies a filter on the respective RSSI to obtain the respective signal strength. In one or more embodiments, the RTLS engine 252 applies a Kalman filter on the respective RSSI to obtain the respective signal strength.

At processing step 1506, the at least one processing unit estimates, based on the respective signal strengths, respective distances between the at least one wireless mobile tag and each respective wireless locating node.

It will be appreciated that the signal strength is used to determine the distance in embodiments using power-based parameters, and in embodiments using signal propagation time measurement parameters or phase difference measurement across frequencies parameters, the distance may be determined based on the signal propagation time measurement parameters and the phase difference measurement parameters without using signal strength.

In some embodiments, where the wireless nodes and mobile tags are equipped with infrared or ultrasonic receivers and transmitters, the at least one processing unit may receive second type of signal data (e.g., infrared signal or ultrasonic signal data), which may indicate if a given tag was detected by a wireless node within a delimited zone, as such signals do not propagate through physical barriers. In combination with the first wireless signal strength parameters, this enables directly discriminating signal strength data from tags not located in the same room as the wireless node, thus optimizing computing resources when calculating distances and proximity.

In one or more embodiments, the RTLS engine 252 estimates the distance between a given mobile tag and each respective wireless node 230 based on the respective signal strength using a path loss exponent in a propagation model. The RTLS engine 252 may use the RSSI at 1 meter in addition to the respective signal strength in the form of a filtered RSSI in a path loss propagation model to calculate the distance estimate.

At processing step 1508, the at least one processing unit classifies, based on the respective estimated distances, a respective proximity of the at least one wireless mobile tag to each respective wireless locating node.

In one or more embodiments, the RTLS engine 252 classifies the proximity of a given mobile tag to each wireless node based on the respective distance being above, equal to and/or below one or more thresholds.

As a non-limiting example, estimated distances below a first threshold (e.g. 2 m) may be classified as very close, estimated distances between a first threshold and a second threshold (e.g., between 2 m and 5 m) may be classified as close, and estimates distances above a third threshold (e.g., 5 m) may be classified as far. The RTLS engine 252 calculates the proximity for each mobile wireless tag.

In some embodiments, the at least one processing unit may transmit, with regard to each wireless locating node, the respective estimated distances and the respective proximity of the at least one wireless mobile tag to each respective wireless locating node.

In one or more other embodiments, the method 1500 comprises processing step 1510.

At processing step 1510, the at least one processing unit outputs, to a user interface, the respective estimated distances and the respective proximity of the at least one wireless mobile tag to each respective wireless locating node.

In one or more embodiments, the respective estimated distances and the respective proximity of the at least one wireless mobile tag to each respective wireless locating node is transmitted to a client device, such as the workstation computer 284 and/or client device 282 for display.

In one or more other embodiments, the respective estimated distances and/or the respective proximity of at least one wireless mobile tag to each respective wireless locating node are transmitted for output into alternative forms (e.g., image, video, audio, etc.)

The method 1500 then ends.

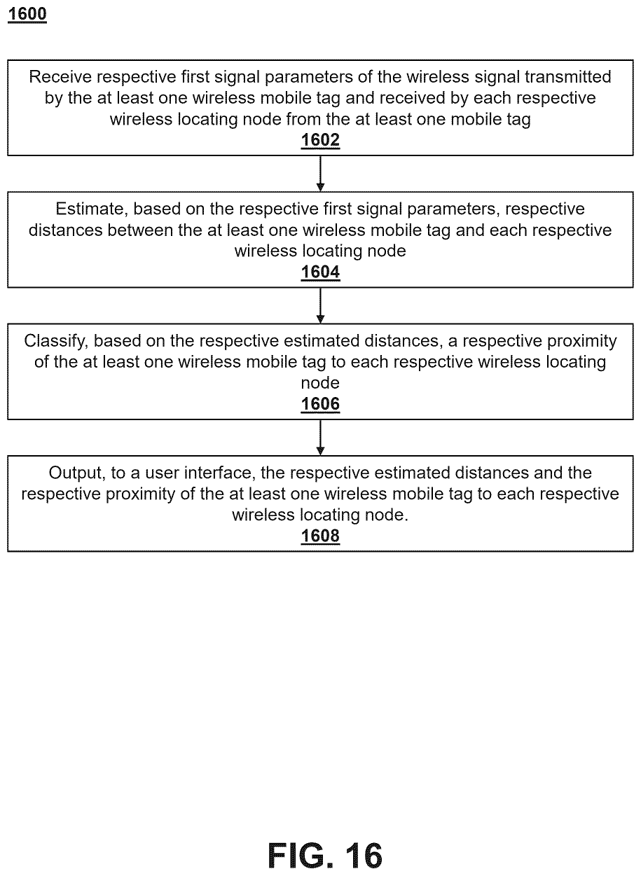

With reference to FIG. 16, there is illustrated a flowchart of a method 1600 of locating wireless mobile tags in accordance with one or more non-limiting embodiments of the present technology. The method 1600 may be executed within the environment of FIG. 2.

The method 1600 is similar to method 1500 of FIG. 15, however the distances are determined based on one of signal propagation time measurement parameters or phase difference measurement across frequencies parameters, without using signal power parameters (e.g., RSSI).

In one or more embodiments, the method 1600 is executed by at least one processing unit. The at least one processing unit is operatively connected to a non-transitory storage medium storing computer-readable instruction thereon. The at least one processing unit, upon executing the computer-readable instructions, is configured to or operable to execute method 1600.

In one or more embodiments, the method 1600 is executed by the RTLS engine 252 executed by the server 240. The server 240 is operatively connected to the plurality of wireless nodes 230 over the communication network 290. The plurality of wireless nodes 230 are configured for receiving wireless signals transmitted by the plurality of wireless tags 220.

The method 1600 begins at processing step 1602.

At processing step 1602, the at least one processing unit receives respective first signal parameters of the wireless signals transmitted by the at least one wireless mobile tag and received by each respective wireless locating node from the at least one mobile tag.

Each of the plurality of wireless tags 220 transmits wireless signals, which are detected by the wireless locating nodes 230 in range. Each wireless locating node 230 in range measures first wireless signal parameters of the wireless signals received by nodes in range.

In one or more alternative embodiments, the first wireless signal parameters may include signal propagation time measurement parameters (time of flight (ToF), and round-trip time (RTP) related parameters including time of departure/arrival, timestamp resolution, clock offset and drift, signal bandwidth and sampling, etc.)

In one or more embodiments, the first wireless signal parameters may include phase difference measurement across frequencies parameters that enable determining room-level distances (e.g., Frequency Domain Phase Difference of Arrival (FD-PDOA), phase-based ranging, Space Domain Phase Difference of Arrival (SD-PDOA), etc.). Phase difference measurement across frequencies parameters may include one or more of: carrier frequency, frequency separation between tones, number of measurements frequencies, phase resolution, signal bandwidth, and update rate.

At processing step 1604, the at least one processing unit estimates, based on the respective first signal parameters, respective distances between the at least one wireless mobile tag and each respective wireless locating node.

In one or more embodiments, the at least one processing unit estimates, based on one of signal propagation time measurement parameters and phase difference measurement across frequencies parameters, the respective distance between the at least one wireless mobile tag and each respective wireless locating node.

In some embodiments, where the wireless nodes and mobile tags are equipped with infrared or ultrasonic receivers and transmitters, the at least one processing unit may receive second type of signal data (e.g., infrared signal or ultrasonic signal data), which may indicate if a given tag was detected by a wireless node within a delimited zone, as such signals do not propagate through physical barriers. In combination with the first wireless signal parameters, this enables directly discriminating signal parameters from tags not located in the same room as the wireless node, thus optimizing computing resources when calculating distances and proximity.

At processing step 1606, the at least one processing unit classifies, based on the respective estimated distances, a respective proximity of the at least one wireless mobile tag to each respective wireless locating node.

In one or more embodiments, the RTLS engine 252 classifies the proximity of a given mobile tag to each wireless node based on the respective distance being above, equal to and/or below one or more thresholds.

As a non-limiting example, estimated distances below a first threshold (e.g. 2 m) may be classified as very close, estimated distances between a first threshold and a second threshold (e.g., between 2 m and 5 m) may be classified as close, and estimates distances above a third threshold (e.g., 5 m) may be classified as far. The RTLS engine 252 calculates the proximity for each mobile wireless tag.

At processing step 1608, the at least one processing unit outputs, to a user interface, the respective estimated distances and the respective proximity of the at least one wireless mobile tag to each respective wireless locating node.

In one or more embodiments, the respective estimated distances and the respective proximity of the at least one wireless mobile tag to each respective wireless locating node is transmitted to a client device, such as the workstation computer 284 and/or client device 282 for display.

The method 1600 then ends.

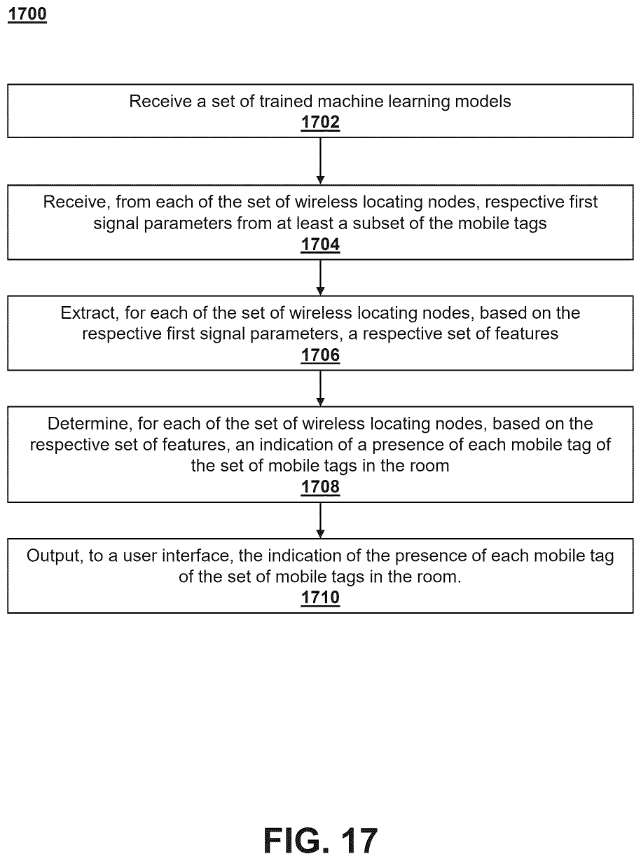

With reference to FIG. 17, there is illustrated a flowchart of a method 1700 of locating wireless mobile tags using a set of trained machine learning models in accordance with one or more non-limiting embodiments of the present disclosure.

The method 1700 is executed by at least one processing unit. The at least one processing unit is operatively connected to a non-transitory storage medium storing computer-readable instruction thereon. The at least one processing unit, upon executing the computer-readable instructions, is configured to or operable to execute method 1700.

The method 1700 begins at processing step 1702. It should be understood that processing 1702 is executed after a training procedure of a set of trained machine learning models, where each machine learning model is associated with a respective wireless locating node in a room of a facility. To train the machine learning (ML) models, each ML model of the set of ML models associated with a respective wireless locating node of the set of wireless locating nodes and is trained on a set of respective first signal parameters for a set of positions of a second set of wireless tags in the facility. Each wireless locating node is associated with a respective room, each of the set of wireless tags is associated with a respective label, where each respective label is an indication of the presence of the wireless in the respective room. The training procedure of the ML models may be performed on a computing device different from the computing device executing the method 1700.

At processing step 1702, the at least one processing unit receives the set of trained ML models. The at least one processing unit may receive the set of trained ML models from another computing device over a communication network, or may receive the ML models from a storage medium.

At processing step 1704, the at least one processing unit receives, from each of the set of wireless locating nodes, respective first signal parameters from at least a subset of the mobile tags.

At processing step 1706, the at least one processing unit extracts, for each of the set of wireless locating nodes, based on the respective first signal parameters, a respective set of features.

The respective set of features have been determined during training of the set of ML models.

At processing step 1708, the at least one processing unit determines, for each of the set of wireless locating nodes, using the set of trained ML models, based on the respective set of features, an indication of a presence of each mobile tag of the set of mobile tags in the room.

At processing step 1710, the at least one processing unit outputs, to a user interface, the indication of the presence of each mobile tag of the set of mobile tags in the room.

The method 1700 then ends.

Any of the embodiments described herein may be combined with one another in various arrangements or subcombinations, unless otherwise specified.

1. A system for locating a mobile tag in real-time in a medical facility, the system comprising:
at least one mobile tag, the at least one mobile tag being configured for:
transmitting a first type of wireless signal comprising a unique identifier;
a set of wireless locating nodes, each wireless locating node being positioned at a respective location in the medical facility, each wireless locating node being configured for:
receiving the first type of wireless signal;
determining, based on the received first type of wireless signal, respective first signal parameters of the at least one mobile tag; and
at least one computing device, the at least one computing device being connected to the set of wireless locating nodes, the at least one computing device being configured for:
receiving the respective first signal parameters of the first type of wireless signal transmitted by the at least one wireless mobile tag and received by each respective wireless locating node from the at least one mobile tag;
determining, based the respective first signal parameters, a respective signal strength of the at least one mobile tag for each respective wireless locating node;
estimating, based on the respective signal strengths, respective distances between the at least one wireless mobile tag and each respective wireless locating node; and
classifying, based on the respective estimated distances, a respective proximity of the at least one wireless mobile tag to each respective wireless locating node.

2. The system of claim 1, wherein the respective first signal parameters comprise a respective Received Signal Strength Indicator (RSSI).

3. The system of claim 2, wherein said determining the respective signal strength for each respective wireless locating node from the at least one mobile tag comprises: applying a Kalman filter on the respective RSSI to obtain the respective signal strength.

4. The system of claim 3, wherein said estimating, based on the respective signal strength, the respective proximity comprises: calculating the respective proximity based on at least the respective signal strength and a path loss exponent of an environment.

5. The system of claim 1, wherein
each wireless locating node of the set of wireless locating nodes is further configured for:
transmitting a second type of wireless signal comprising a respective indication of a respective wireless node identifier; wherein
the at least one wireless mobile tag is further configured for:
receiving, from a given wireless node of the set of wireless nodes, a second type of wireless signal comprising an indication of the given wireless node identifier;
transmitting in the first type of wireless signal, the indication of the given wireless node identifier; and wherein
the at least one computing device is further configured for:
determining, based on the respective proximities and the given wireless node identifier, a location of the given wireless node.

6. The system of claim 5, wherein the first type of wireless signal comprises a Bluetooth signal and the second type of wireless signal comprises an infrared signal.

7. The system of claim 1, wherein the at least one mobile tag is integrated into a wristband.

8. The system of claim 1, wherein the at least one mobile tag is attachable to an article of clothing.

9. The system of claim 1 wherein the at least one mobile tag further comprises an accelerometer, the at least one mobile tag being configured for transmitting accelerometer motion data.

10. The system of claim 9, wherein the at least one computing device is further configured for:
receiving the accelerometer motion data; and
detecting, based on the accelerometer motion data, a position of the patient.

11. A method for approximately locating a wireless tag in a room of a facility, the method being executed by at least one processing unit, the at least one processing unit being in communication with:
a set of wireless locating nodes, each wireless locating node being positioned at a respective location in the facility, each wireless locating node being configured for transmitting and receiving a wireless signal,
the at least one wireless mobile tag, the at least one wireless mobile tag being configured for receiving a wireless signal,
the method comprising:
receiving respective first signal parameters of the wireless signal transmitted by the at least one wireless mobile tag and received by each respective wireless locating node from the at least one mobile tag;
determining, based the respective first signal parameters, a respective signal strength for each respective wireless locating node from the at least one mobile tag;
estimating, based on the respective signal strengths, respective distances between the at least one wireless mobile tag and each respective wireless locating node;
classifying, based on the respective estimated distances, a respective proximity of the at least one wireless mobile tag to each respective wireless locating node; and
outputting, to a user interface, the respective estimated distances and the respective proximity of the at least one wireless mobile tag to each respective wireless locating node.

12. The method of claim 11, wherein the respective signal parameters comprise a respective Received Signal Strength Indicator (RSSI).

13. The method of claim 12, wherein said determining the respective signal strength for each respective wireless locating node from the at least one mobile tag comprises applying a Kalman filter on the respective RSSI to obtain the respective signal strength.

14. The method of claim 13, wherein said estimating, based on the respective signal strength, the respective distances comprises: calculating the respective proximity based on at least the respective signal strength and a path loss exponent of the environment.

15. The method of claim 14, wherein the respective signal parameters further comprises a respective RSSI at 1 meter, and wherein said calculating the respective proximity is further based on the respective RSSI at 1 meter.

16. The method of claim 11 wherein the respective proximity comprises one of: very close, close and far.

17. The method of claim 11, wherein the wireless signal comprises a Bluetooth signal.

18. The method of claim 17, further comprising: associating, by using a rule-based procedure, based on the respective estimated distances and the respective proximity of the at least one wireless mobile tag to each respective wireless locating node, a given wireless node of the set of wireless nodes with the given wireless node.

19. A method for predicting presence of a wireless tag in a room of a facility using a set of machine learning (ML) models, the method being executed by at least one processing unit, the at least one processing unit being connected to:
a set of wireless locating nodes, each wireless locating node being positioned at a respective location in the facility, each wireless locating node being configured for transmitting and receiving wireless signals from a set of mobile tags,
the at least one processing unit having access to the set of ML models, each ML model being associated with a respective wireless locating node of the set of wireless locating nodes, each ML model having been trained for predicting the presence of a given wireless tag,
the method comprising:
receiving, from each of the set of wireless locating nodes, respective first signal parameters from at least a subset of the mobile tags;
extracting, for each of the set of wireless locating nodes, based on the respective first signal parameters, a respective set of features;
determining, for each of the set of wireless locating nodes, based on the respective set of features, an indication of a presence of each mobile tag of the set of mobile tags in the room; and
outputting, to a user interface, the indication of the presence of each mobile tag of the set of mobile tags in the room.

20. The method of claim 19, further comprising, prior to said receiving, from each of the set of wireless locating nodes, the respective first signal parameters from at least a subset of the mobile tags:
training each ML model of the set of ML models associated with a respective wireless locating node of the set of wireless locating nodes on a set of respective first signal parameters for a set of positions of a second set of wireless tags in the facility, each wireless locating node being associated with a respective room, each of the second set of wireless tags being associated with a respective label, the respective label being indicative of the presence of the wireless in the respective room.
